SEQUENCE LISTING (12 sequences)

SEQ ID NO: 1 - DNA, 30 bp, Artificial
  Mammalian-derived
gtaattgcggcaagaagaattgtttctgtc

SEQ ID NO: 2 - DNA, 30 bp, artificial
  Mammalian-derived
gtaattgcggcaggaggaattgtttctgtc

SEQ ID NO: 3 - DNA, 30 bp, artificial
  Mammalian-derived
ggcaagagacaccaaagacactaccagcat

SEQ ID NO: 4 - DNA, 27 bp, artificial
  Mammalian-derived
tcctgagcaatacctaacgaacaaata

SEQ ID NO: 5 - DNA, 20 bp, artificial
  Mammalian-derived
catctccttggtgctcaacc

SEQ ID NO: 6 - DNA, 20 bp, artificial
  Mammalian-derived
ctgaagtcgacttggcttgg

SEQ ID NO: 7 - DNA, 21 bp, Artificial
  Mammalian-derived
ctcagatagtggccagaatgc

SEQ ID NO: 8 - DNA, 20 bp, artificial
  Mammalian-derived
ttgtccaggtgactccaagg

SEQ ID NO: 9 - DNA, 25 bp, artificial
  Mammalian-derived
cgtccgagcccagaaagatgaggtc

SEQ ID NO: 10 - DNA, 19 bp, artificial
  Mammalian-derived
agaggcgcacgtgagacac

SEQ ID NO: 11 - DNA, 19 bp, artificial
  Mammalian-derived
tgaagacaatgaagatgtt

SEQ ID NO: 12 - DNA, 25 bp, artificial
  Mammalian-derived
tttcttttctatgtgctgttggtga

(19) United States
(12) Patent Application Publication
(10) Pub. No.: US 2011/0144182 A1
(43) Pub. Date: Jun. 16, 2011

(54) TREATMENT OF SURGICAL ADHESIONS

(76) Inventors: David Lawrence Becker, Abbots Langley (GB); Colin Richard Green, Epson (NZ); Bradford James Duft, Rancho Santa Fe, CA (US)

(21) Appl. No.: 12/809,933

(22) PCT Filed: Dec. 22, 2008
(86) PCT No.: PCT/US08/14024

§ 371 Date: Feb. 14, 2011

Related U.S. Application Data
(62) Division of application No. 61/008,748, filed on Dec. 21, 2007.

(51) Int. Cl. A61K 31/7088; A61K 31/713; A61K 31/7105; A61K 31/711; C07H 21/00; A61P 41/00; A61P 17/00

(52) U.S. Cl. 514/44 A; 536/24.5

(57) ABSTRACT

Connexin modulation for the treatment of surgical adhesions, and associated methods, compositions, and articles.

This application is a National Stage Application under 35 U.S.C. §371 of International Application No. PCT/US2008/014024, filed on Dec. 22, 2008 which claims the benefit of priority to U.S. Provisional Application No. 61/008,748 filed on Dec. 21, 2007. The disclosures of both are incorporated herein by reference.

### FIELD

The inventions relate to adhesions, more particularly surgical adhesions, and methods of treatment thereof, as well as compositions, formulations, articles and kits, and delivery devices comprising such compositions.

### BACKGROUND

The following includes information that may be useful in understanding the present invention. It is not an admission that any of the information provided herein is prior art, or relevant, to the presently described or claimed inventions, or that any publication or document that is specifically or implicitly referenced is prior art.

In humans and other mammals wound injury triggers an organized complex cascade of cellular and biochemical events that will in most cases result in a healed wound. An ideally healed wound is one that restores normal anatomical structure, function, and appearance at the cellular, tissue, organ, and organism levels. Wound healing, whether initiated by surgery, disease, trauma, microbes or foreign materials, proceeds via a complex process encompassing a number of overlapping phases, including inflammation, epithelialization, angiogenesis and matrix deposition. Normally, these processes lead to a mature wound and a certain degree of scar formation.

Adhesion formation is a process in which bodily tissues that are normally separate become connected by scar tissue. Adhesions most commonly result from surgical incision, abrasion, or trauma. Adhesions can form following most any type of surgery, but develop with the highest frequency following general abdominal, gynecologic, orthopedic, and cardiac surgeries. It has been reported that following abdominal surgery the incidence of peritoneal adhesion formation may be as high as 90%. See U.S. Pat. No. 6,613,325. The incidence of adhesion formation is also thought to be as high as 90% in patients that have undergone multiple surgeries. Post operative intraperitoneal and pelvic adhesions represent a major problem in patients recovering from surgery in the abdominal cavity, where there is a tendency for adhesions to form between the affected tissues. See U.S. Pat. No. 5,002,551. The pervasiveness of this problem also has severe economic consequences.

Although adhesions occur most commonly following surgery, adhesions may also occur from tissue damage other than surgery, including traumatic injury, inflammatory disease, intraperitoneal chemotherapy and radiation therapy. Amongst other complications, the presence of surgical adhesions may be associated with pain, discomfort, and female infertility resulting from gynecological surgery. Intestinal obstructions, for example, are a complication that results from surgical adhesions. Adhesions are also reported to be a leading cause of bowel obstruction and infertility, and related complications include chronic pelvic pain, urethral obstruction and voiding dysfunction. See U.S. Pat. No. 6,689,803. Adhesion formation may result from injury to the peritoneum, which in turn may cause the site of injury or trauma to become inflamed. Although inflammation is a part of the healing process, it can contribute to adhesion formation by contributing to the development of fibrous bands of scar tissue. Through a process called fibrinolysis, the fibrin bands eventually dissolve. However, where fibrin bands do not dissolve, they can develop into proliferating adhesions that connect and bind to organs and tissues that are normally separate. It has been reported that excess production and deposition of the extracellular matrix may be a key factor in producing tissue fibrosis throughout the body including the development of peritoneal adhesions (see U.S. Pat. No. 6,841,153).

Various approaches for the prevention of adhesion formation have been reported. See Dizerega, G. S. & Rodgers, K. E., “Prevention of Postoperative Adhesions,” in “The Peritoneum,” Dizerega, G. S. & Rodgers, K. E., eds., Springer-Verlang, New York, pp. 307-369 (1992). General categories of treatment for adhesions that have been reported, include: 1) prevention of fibrin deposition in the peritoneal exudate, 2) reduction of local tissue inflammation; and 3) removal of fibrin deposits. Id. However, despite years of research it has been reported that very few products for the prevention of post-operative adhesions have resulted. Johns, A., Human Reproductive Update, 7(6):577-579 (2001). Meanwhile, the medical problems associated with surgical adhesions are becoming more serious because there is a general rise in repeat surgical procedures for a number of disorders. Thus, there is a vital need for the development of compounds and methods for preventing surgical adhesions and mitigating the complications they cause.

Gap junctions are cell membrane structures that facilitate direct cell-cell communication. A gap junction channel is formed of two connexins (hemichannels), each composed of six connexin subunits. Each hexameric connexin docks with a connexin in the opposing membrane to form a single gap junction. Gap junction channels are reported to be found throughout the body. Tissue such as the corneal epithelium, for example, has six to eight cell layers, yet is reported to expresses different gap junction channels in different layers with connexin 43 in the basal layer and connexin 26 from the basal to middle wing cell layers. In general, connexins are a family of proteins, commonly named according to their molecular weight or classified on a phylogenetic basis into alpha, beta, and gamma subclasses. At least 20 human and 19 murine isoforms have been identified. Different tissues and cell types are reported to have characteristic patterns of connexin protein expression and tissues such as cornea have been shown to alter connexin protein expression pattern following injury or transplantation (Qui, C. et al., (2003) Current Biology, 13:1967-1703; Brander et al., (2004), J. Invest Dermatol. 122:1310-20).

It has been reported that abnormal connexin function may be linked to certain disease states (e.g. heart diseases) (A. C. de Carvalho, et al., J Cardiovasc Electrophysiol 1994, 5 686). In certain connexin proteins, alterations in the turnover and trafficking properties may be induced by the addition exogenous agents which may affect the level of gap junctional intercellular communication (Darrow, B. J., et al. (1995). Circ Res 76: 381; Lin R, et al. (2001) J Cell Biol 154(4):815). Antisense technology has been reported for the modulation of the expression for genes implicated in viral, fungal and metabolic diseases. See, e.g., U.S. Pat. No. 5,166,195, (oligonucleotide inhibitors of HIV), U.S. Pat. No. 5,004,810 (oligomers for hybridizing to herpes simplex virus Vmw65 mRNA and inhibiting replication). See also U.S. Pat. No. 7,098,190 to Becker et al. (formulations comprising antisense nucleotides to connexins). Peptide inhibitors (including mimetic peptides) of gap junctions and hemichannels have been reported. See for example Berthoud, V. M. et al., Am J. Physiol. Lung Cell Mol. Physiol. 279: L619-L622 (2000); Evans, W. H. and Boitano, S. Biochem. Soc. Trans. 29: 606-612, and De Vriese A. S., et al. Kidney Int. 61: 177-185 (2001). See also Becker and Green PCT/US06/04131 (“Anti-connexin compounds and uses thereof”).

### BRIEF SUMMARY

The inventions described and claimed herein have many attributes and embodiments including, but not limited to, those set forth or described or referenced in this Brief Summary. It is not intended to be all-inclusive and the inventions described and claimed herein are not limited to or by the features or embodiments identified in this Brief Summary, which is included for purposes of illustration only and not restriction.

The present invention provides methods for treating, reducing the incidence of and/or preventing surgical adhesions.

In one aspect, the invention relates to a method of preventing or decreasing adhesions, comprising administration of an anti-connexin polynucleotide to a subject in need thereof.

The invention also relates to a method of preventing or decreasing post-surgical adhesions in a subject which comprises administering an effective amount of an anti-connexin polynucleotide to the patient at a site of surgery. In one embodiment the anti-connexin polynucleotide is administered at the site of a surgical incision. In one embodiment the anti-connexin polynucleotide is administered during and/or after surgery. In one embodiment the anti-connexin polynucleotide is effective, in whole or in part, to (1) downregulate expression of a connexin protein (2) inhibit intercellular communication by decreasing gap junction formation, (3) prevent or reduce surgical adhesions at a site of the surgery or surgical repair.

It also relates to a method of preventing or decreasing formation of secondary surgical adhesion, comprising administration of an effective amount of an anti-connexin polynucleotide to subject a following a procedure to repair an adhesion. In one embodiment the procedure is a separation or release procedure. In one embodiment the anti-connexin polynucleotide is administered at the site of surgical incision. In one embodiment the anti-connexin polynucleotide is administered during and/or after surgery. In one embodiment the anti-connexin polynucleotide is effective, in whole or in part, to (1) downregulate expression of a connexin protein (2) inhibit intercellular communication by decreasing gap junction formation, (3) prevent or reduce secondary surgical adhesions at a site of the surgery or surgical repair.

In certain embodiments, the anti-connexin polynucleotide is administered to epithelial, connective, muscle, and nerve tissue or other tissue exposed or wounded during surgery or as a result of trauma. In one embodiment, the anti-connexin polynucleotide is administered topically. In other embodiments, the anti-connexin polynucleotide is implanted or instilled or injected.

The invention also relates to a method of preventing or decreasing formation of adhesions in a patient at risk thereof, which comprises administering a therapeutically effective amount of an anti-connexin polynucleotide to said patient. In one embodiment the patient has had surgery. In one embodiment, the adhesion is a surgical adhesion. In one embodiment the patient has suffered an injury or trauma.

In one embodiment the method of treatment further comprises administration of one or more therapeutic agents, agents useful for wound healing, and/or anti-microtubule agents.

According to certain embodiment the subject is a mammal. In one embodiment the mammal is a human. In another embodiment, the subject is an animal or a bird. Birds include pets and poultry. Animals include swine, cattle and sports animals and pets such as horses, dogs and cats.

The invention also relates to a method for preventing or reducing the formation of surgical adhesions in a subject comprising administering an effective amount of therapeutic formulation containing, as an active ingredient, an anti-connexin polynucleotide.

The invention further relates to a method for reducing or preventing adhesions in a patient comprising exposing tissue which has been subjected to tissue damage and is at risk for the formation of adhesions a pharmaceutical composition comprising an anti-connexin polynucleotide and a pharmaceutical acceptable carrier.

Thus, the invention also relates to pharmaceutical compositions and formulations useful for treating or preventing adhesions, including for example surgical adhesions.

In one aspect, the invention provides a pharmaceutical composition useful for treating or preventing adhesions comprises one or more anti-connexin polynucleotides (e.g. connexin antisense polynucleotides). Preferably, the pharmaceutical composition further comprises a pharmaceutically acceptable carrier, diluent or excipient. For example, the inventions include pharmaceutical compositions useful for treating or preventing adhesions comprising (a) a therapeutically effect amount of a pharmaceutically acceptable connexin antisense polynucleotide and (b) a pharmaceutically acceptable carrier or diluent.

The invention also includes pharmaceutical compositions useful for treating or preventing adhesions comprising (a) a therapeutically effective amount of an anti-connexin polynucleotide, and (b) a therapeutically effective amount of one or more therapeutic agents. The invention includes pharmaceutical compositions useful for treating or preventing adhesions comprising (a) a therapeutically effective amount of an anti-connexin polynucleotide, and (b) a therapeutically effective amount of one or more and/or agents useful in wound healing. The invention includes pharmaceutical compositions useful for treating or preventing adhesions comprising (a) a therapeutically effective amount of an anti-connexin polynucleotide, and (b) a therapeutically effective amount of one or more anti-microtubule agents. Preferably, the pharmaceutical compositions further comprise a pharmaceutically acceptable carrier, diluent or excipient.

Thus, for example, pharmaceutical compositions useful for treating or preventing adhesions are also provided in the form of a combined preparation, for example, as an admixture of one or more anti-connexin polynucleotides and one or more other agents useful for wound healing, e.g., growth factors that are effective in promoting or improving wound healing, such as platelet derived growth factor, epidermal growth factor, fibroblast growth factor (e.g., FGF2), vascular endothelial growth factor, and transforming growth factor β3, and/or cytokines that are effective in promoting or improving wound healing, such as IL-7 and IL-10, and/or other agents that are effective in promoting or improving wound healing, such as IGF (e.g., IGF-1) and IGFBP (e.g., IGFBP-2).

The term “a combined preparation” includes a “kit of parts” in the sense that the combination partners as defined above can be dosed independently or by use of different fixed combinations with distinguished amounts of the combination partners (a) and (b), i.e. simultaneously, separately or sequentially. The parts of the kit can then, for example, be administered simultaneously or chronologically staggered, that is at different time points and with equal or different time intervals for any part of the kit of parts.

In a preferred embodiment, the administration of a combined preparation will have fewer administration time points and/or increased time intervals between administrations as a result of such combined use.

In another aspect, the invention includes formulations comprising an effective amount of one or more pharmaceutically acceptable connexin antisense polynucleotides formulated in a delayed release preparation, a slow release preparation, an extended release preparation, a controlled release preparation, and/or in a repeat action preparation to a subject suffering from or at risk of forming an adhesion.

In a further aspect, the invention includes transdermal patches, dressings, pads, wraps, matrices and bandages capable of being adhered or otherwise associated with the skin of a subject, said articles being capable of delivering a therapeutically effective amount of one or more pharmaceutically acceptable anti-connexin polynucleotides, e.g., connexin antisense polynucleotides to a patient to prevent or retard the formation of an adhesion.

The invention includes devices useful for treating or preventing adhesions containing therapeutically effective amounts of one or more pharmaceutically acceptable anti-connexin polynucleotides, e.g., connexin antisense polynucleotides, for example, a rate-controlling membrane enclosing a drug reservoir and a monolithic matrix device. These devices may be employed for the treatment of subjects in need thereof as disclosed herein. Suitably the wound dressing or matrix is provided including the form of a solid substrate with an anti-connexin polynucleotide, e.g., a connexin antisense polynucleotide, either alone or in combination with one or more therapeutic agents and/or agents useful for wound healing, dispersed on or in the solid substrate. In one embodiment the pharmaceutical product of the invention is provided in combination with a wound dressing or wound healing promoting matrix. Preferred anti-connexin polynucleotides and connexin antisense polynucleotides are anti-connexin 43 polynucleotides and connexin 43 antisense polynucleotides.

Pharmaceutical compositions useful for treating or preventing adhesions are provided for combined, simultaneous, separate sequential or sustained administration. In one embodiment, a composition comprising one or more anti-connexin polynucleotides is administered at or about the same time as one or more therapeutic agents, agents useful for wound healing and/or anti-microtubule agents.

In certain embodiments, the anti-connexin polynucleotide decreases connexin protein expression, wherein said connexin is selected from the group consisting of connexin 26, connexin 30, connexin 30.3, connexin 31.1, connexin 32, connexin 36, connexin 37, connexin 40, connexin 40.1, connexin 43, connexin 45, connexin 46 and connexin 46.6. In a preferred embodiment, the anti-connexin polynucleotide decreases expression of connexion 43. In another preferred embodiment, the connexin is a human connexin.

Examples of a connexin antisense polynucleotide include, for example, an anti-connexin oligodeoxynucleotide (ODN), including antisense (including modified and unmodified backbone antisense; e.g., a DNA antisense polynucleotide that binds to a connexin mRNA), RNAi, and siRNA polynucleotides.

Suitable connexin antisense polynucleotides include for example, antisense ODNs against connexin 43 (Cx43), connexin 26 (Cx26), connexin 37 (Cx37), connexin 30 (Cx30), connexin 31.1 (Cx31.1) and connexin 32 (Cx32). In certain embodiments, suitable compositions include multiple connexin antisense polynucleotides in combination, including for example, polynucleotides targeting Cx 43, 26, 30, and 31.1. Preferred connexin antisense polynucleotides target connexin 43.

Conveniently, the oligodeoxynucleotide to connexin 43 is selected from: GTA ATT GCG GCA AGA AGA ATT GTT TCT GTC (SEQ.ID.NO:1); GTA ATT GCG GCA GGA GGA ATT GTT TCT GTC (SEQ.ID.NO:2); GGC AAG AGA CAC CAA AGA CAC TAC CAG CAT (SEQ.ID.NO:3), a polynucleotide having at least about 70 percent homology with SEQ.ID.NOS: 1, 2, or 3 or a polynucleotide which hybridizes to connexin 43 mRNA under conditions of medium to high stringency.

The invention also relates to a method to evaluate the anti-adhesion activity of an anti-connexin polynucleotide, comprising contacting cells at risk of forming an adhesion with an anti-connexin polynucleotide, and determining the anti-adhesion effect of said an anti-connexin polynucleotide. In one embodiment, said method is carried out in vitro. In another embodiment, said method is carried out in vivo.

The invention further relates to an article of manufacture useful for treating or preventing adhesions comprising: (a) a pharmaceutical composition having (i) an anti-connexin polynucleotide, and (ii) a pharmaceutically acceptable carrier, and (b) instructions for administering the pharmaceutical composition to a patient having surgery, or otherwise at risk of having an adhesion. In certain embodiments, the instructions describe administration of the pharmaceutical composition to the patient to treat surgical adhesions after a surgical procedure and administering the pharmaceutical composition in a quantity sufficient to prevent or reduce surgical adhesions at a site of the procedure or a resulting wound. Preferred anti-connexin polynucleotides and connexin antisense polynucleotides are anti-connexin 43 polynucleotides and connexin 43 antisense polynucleotides. In one embodiment, the composition further comprises a therapeutically effective amount of one or more therapeutic agents, agents useful for wound healing and/or anti-microtubule agents. In one embodiment, the article of manufacture additionally comprises a composition containing a therapeutically effective amount of one or more therapeutic agents, agents useful for wound healing and/or anti-microtubule agents.

The invention also further relates to a method of making an article of manufacture useful for treating or preventing adhesions, comprising (2) a vessel containing a therapeutically effective amount of an anti-connexin polynucleotide, and (ii) a pharmaceutically acceptable carrier, and (b) instructions for treating a patient having or at risk of having an adhesion, e.g., as a result of surgery, injury or trauma. Preferred anti-connexin polynucleotides and connexin antisense polynucleotides are anti-connexin 43 polynucleotides and connexin 43 antisense polynucleotides. In one embodiment, the composition further comprises a therapeutically effective amount of one or more therapeutic agents, agents useful for wound healing and/or anti-microtubule agents. In one embodiment, the article of manufacture additionally comprises a composition containing a therapeutically effective amount of one or more therapeutic agents, agents useful for wound healing and/or anti-microtubule agents.

The invention also further relates to a method of making an article of manufacture useful for treating or preventing adhesions, comprising (2) a vessel containing a therapeutically effective amount of an anti-connexin polynucleotide, and (ii) a pharmaceutically acceptable carrier, and (b) instructions for treating a patient having or at risk of forming a secondary adhesion as a result of a corrective surgical procedures, e.g. a release or separation procedure. Preferred anti-connexin polynucleotides and connexin antisense polynucleotides are anti-connexin 43 polynucleotides and connexin 43 antisense polynucleotides. In one embodiment, the composition further comprises a therapeutically effective amount of one or more therapeutic agents, agents useful for wound healing and/or anti-microtubule agents. In one embodiment, the article of manufacture additionally comprises a composition containing a therapeutically effective amount of one or more therapeutic agents, agents useful for wound healing and/or anti-microtubule agents.

The invention further relates to a method of making an article of manufacture useful for treating or preventing adhesions, comprising packaging material containing one or more dosage forms containing (i) an anti-connexin polynucleotide, and (ii) a pharmaceutically acceptable carrier, and (b) labeling instructions for treating a patient having or at risk of having adhesions by administering the pharmaceutical composition to a patient. In certain embodiments, the instructions describe administration of dosage form to the patient to treat adhesions or secondary adhesions in a patient undergoing surgery.

### DETAILED DESCRIPTION

### Definitions

As used herein, “subject” refers to any mammals, including humans, domestic and farm animals, and zoo, sports, or pet animals, such as dogs, horses, cats, sheep, pigs, cows, etc. The preferred mammal herein is a human, including adults, children, and the elderly.

As used herein, “preventing” means preventing in whole or in part, or ameliorating or controlling.

As used herein, a “therapeutically effective amount” or “effective amount” in reference to the polynucleotides or compositions of the instant invention refers to the amount sufficient to induce a desired biological, pharmaceutical, or therapeutic result. That result can be alleviation of the signs, symptoms, or causes of a disease or disorder or condition, or any other desired alteration of a biological system. In the present invention, the result will involve preventing, retarding or reducing the incidence or severity of and/or decreasing the formation of adhesions, surgical adhesions, and/or secondary surgical adhesions, in whole or in part.

As used herein, the term “treating” refers to both therapeutic treatment and prophylactic or preventative measures. Those in need of treatment include those already with an adhesion as well as those prone to having an adhesion or those in which an adhesion is to be prevented.

As used herein, “simultaneously” is used to mean that the one or more anti-connexin polynucleotides, e.g., connexin 43 antisense polynucleotides, alone or in combination with one or more therapeutic agents, agents useful for wound healing and/or anti-microtubule agents are administered concurrently, whereas the term “in combination” is used to mean they are administered, if not simultaneously or in physical combination, then “sequentially” within a timeframe that they both are available to act therapeutically. Thus, administration “sequentially” may permit one polynucleotide or agent to be administered within minutes (for example, 1, 2, 3, 4, 5, 10, 15, 20, 25, 30) minutes or a matter of 1-24 hours, 1-7 days, or 1-4 weeks or months after another polynucleotide or agent provided that both the one or more anti-connexin polynucleotides and one or more therapeutic agent, agents useful in wound healing and/or microtubule agents are concurrently present in effective amounts. The time delay between administration of the components will vary depending on the exact nature of the components, the interaction there between, and their respective half-lives.

As used herein, an “anti-connexin polynucleotide” decreases or inhibits expression of connexin mRNA and/or protein. Anti-connexin polynucleotides include, without limitation, antisense compounds such as antisense polynucleotides, other polynucleotides (such as polynucleotides having siRNA or ribozyme functions). Suitable examples of an anti-connexin polynucleotide include an antisense polynucleotide to a connexin. Accordingly, suitable anti-connexin polynucleotides include, for example, antisense polynucleotides (e.g., connexin 43 antisense polynucleotides) that modulate expression or activity of connexins and gap junctions in selected tissues, cells, and subjects. Exemplary anti-connexin polynucleotides are further described herein.

### Surgical Adhesions

Within other aspects of the invention, methods are provided for treating, reducing the incidence or severity of, and/or preventing or retarding adhesions, surgical adhesions and/or secondary surgical adhesions by administering to a patient an anti-connexin polynucleotide.

As noted herein, surgical adhesion formation is a complex process in which bodily tissues that are normally separate grow together. For example, post-operative adhesions have been reported to occur in about 60% to 90% of patients undergoing major gynecological surgery. Surgical trauma as a result of tissue (e.g. epithelial, connective, muscle, and nerve tissue) drying, ischemia, thermal injury, infection or the presence of a foreign body, has long been recognized as a stimulus for tissue adhesion formation. These adhesions are a major cause of failed surgical therapy and are the leading cause of bowel obstruction and infertility. Other adhesion-treated complications include chronic pelvic pain, urethral obstruction and voiding dysfunction.

Generally, adhesion formation is an inflammatory reaction in which factors are released, increasing vascular permeability and resulting in fibrinogen influx and fibrin deposition. This deposition forms a matrix that bridges the abutting tissues. Fibroblasts accumulate, attach to the matrix, deposit collagen and induce angiogenesis. If this cascade of events can be prevented within 4 to 5 days following surgery, adhesion formation can be inhibited.

Secondary surgical adhesions may also form as a result of a corrective surgical procedure designed to correct and existing adhesion. The procedure may be a release or separation procedure.

A wide variety of animal models may be utilized in order to assess a particular therapeutic composition or treatment regimen for its therapeutic potential. Briefly, peritoneal adhesions have been observed to occur in animals as a result of inflicted severe damage which usually involves two adjacent surfaces. Injuries may be mechanical, due to ischemia or as a result of the introduction of foreign material. Mechanical injuries include crushing of the bowel (Choate et al., Arch. Surg. 88:249-254, 1964) and stripping or scrubbing away the outer layers of bowel wall (Gustaysson et al., Acta Chir. Scand 109:327-333, 1955). Dividing major vessels to loops of the intestine induces ischemia (James et al., J. Path. Bact. 90:279-287, 1965). Foreign material that may be introduced into the area includes talcum (Green et al., Proc. Soc Exp. Biol. Med. 133:544-550, 1970), gauze sponges (Lehman and Boys, Ann. Surg 111:427435, 1940), toxic chemicals (Chancy, Arch. Surg. 60:1151-1153, 1950), bacteria (Moin et al. Am. J. Med. Sci. 250:675-679, 1965) and feces (Jackson, Surgery 44:507-518, 1958).

Presently, typical animal models to evaluate prevention of formation of adhesions include the rabbit uterine horn model which involves the abrasion of the rabbit uterus (Linsky et al., J. Reprod. Med. 32(1): 17-20, 1987), the rabbit uterine horn, devascularization modification model which involves abrasion and devascularization of the uterus (Wiseman et al, J. Invest Surg. 7:527-532, 1994) and the rabbit cecal sidewall model which involves the excision of a patch of parietal peritoneum plus the abrasion of the cecum (Wiseman and Johns, Fertil SteriL Suppl: 25S, 1993). Those and other reported evaluation models are described herein.

### Anti-Connexin Polynucleotides

Anti-connexin polynucleotides include connexin antisense polynucleotides as well as polynucleotides which have functionalities which enable them to downregulate or inhibit connexin expression (for example, by downregulation of mRNA transcription or translation). In the case of downregulation, this will have the effect of reducing direct cell-cell communication by gap junctions at the site at which connexin expression is down-regulated.

Suitable anti-connexin polynucleotides include RNAi polynucleotides and siRNA polynucleotides.

Synthesis of antisense polynucleotides and other anti-connexin polynucleotides such as RNAi, siRNA, and ribozyme polynucleotides as well as polynucleotides having modified and mixed backbones is known to those of skill in the art. See e.g. Stein C. A. and Krieg A. M. (eds), Applied Antisense Oligonucleotide Technology, 1998 (Wiley-Liss).

According to one aspect, the downregulation of connexin expression may be based generally upon the antisense approach using antisense polynucleotides (such as DNA or RNA polynucleotides), and more particularly upon the use of antisense oligodeoxynucleotides (ODN). These polynucleotides (e.g., ODN) target the connexin protein (s) to be downregulated. Typically the polynucleotides are single stranded, but may be double stranded.

The antisense polynucleotide may inhibit transcription and/or translation of a connexin. Preferably the polynucleotide is a specific inhibitor of transcription and/or translation from the connexin gene or mRNA, and does not inhibit transcription and/or translation from other genes or mRNAs. The product may bind to the connexin gene or mRNA either (i) 5′ to the coding sequence, and/or (ii) to the coding sequence, and/or (iii) 3′ to the coding sequence.

The antisense polynucleotide is generally antisense to a connexin mRNA. Such a polynucleotide may be capable of hybridizing to the connexin mRNA and may thus inhibit the expression of connexin by interfering with one or more aspects of connexin mRNA metabolism including transcription, mRNA processing, mRNA transport from the nucleus, translation or mRNA degradation. The antisense polynucleotide typically hybridizes to the connexin mRNA to form a duplex which can cause direct inhibition of translation and/or destabilization of the mRNA. Such a duplex may be susceptible to degradation by nucleases.

The antisense polynucleotide may hybridize to all or part of the connexin mRNA. Typically the antisense polynucleotide hybridizes to the ribosome binding region or the coding region of the connexin mRNA. The polynucleotide may be complementary to all of or a region of the connexin mRNA. For example, the polynucleotide may be the exact complement of all or a part of connexin mRNA. However, absolute complementarity is not required and polynucleotides which have sufficient complementarity to fowl a duplex having a melting temperature of greater than about 20° C., 30° C. or 40° C. under physiological conditions are particularly suitable for use in the present invention.

Thus the polynucleotide is typically a homologue of a sequence complementary to the mRNA. The polynucleotide may be a polynucleotide which hybridizes to the connexin mRNA under conditions of medium to high stringency such as 0.03M sodium chloride and 0.03M sodium citrate at from about 50° C. to about 60° C.

For certain aspects, suitable polynucleotides are typically from about 6 to 40 nucleotides in length. Preferably a polynucleotide may be from about 12 to about 35 nucleotides in length, or alternatively from about 12 to about 20 nucleotides in length or more preferably from about 18 to about 32 nucleotides in length. According to an alternative aspect, the polynucleotide may be at least about 40, for example at least about 60 or at least about 80, nucleotides in length and up to about 100, about 200, about 300, about 400, about 500, about 1000, about 2000 or about 3000 or more nucleotides in length.

The connexin protein or proteins targeted by the polynucleotide will be dependent upon the site at which downregulation is to be effected. This reflects the non-uniform make-up of gap junction(s) at different sites throughout the body in terms of connexin sub-unit composition. The connexin is a connexin that naturally occurs in a human or animal in one aspect or naturally occurs in the tissue in which connexin expression or activity is to be decreased. The connexin gene (including coding sequence) generally has homology with the coding sequence of one or more of the specific connexins mentioned herein, such as homology with the connexin 43 coding sequence shown in Table 2. The connexin is typically an α or β connexin. Preferably the connexin is an α connexin and is expressed in the tissue to be treated.

Some connexin proteins are however more ubiquitous than others in terms of distribution in tissue. One of the most widespread is connexin 43. Polynucleotides targeted to connexin 43 are particularly suitable for use in the present invention. In other aspects other connexins are targeted.

In one preferred aspect, the antisense polynucleotides are targeted to the mRNA of one connexin protein only. Most preferably, this connexin protein is connexin 43. In another aspect, connexin protein is connexin 26, 30, 31.1, 32, 36, 37, 40, or 45. In other aspects, the connexin protein is connexin 30.3, 31, 40.1, or 46.6.

It is also contemplated that polynucleotides targeted to separate connexin proteins be used in combination (for example 1, 2, 3, 4 or more different connexins may be targeted). For example, polynucleotides targeted to connexin 43, and one or more other members of the connexin family (such as connexin 26, 30, 30.3, 31.1, 32, 36, 37, 40, 40.1, 45, and 46.6) can be used in combination.

Alternatively, the antisense polynucleotides may be part of compositions which may comprise polynucleotides to more than one connexin protein. Preferably, one of the connexin proteins to which polynucleotides are directed is connexin 43. Other connexin proteins to which oligodeoxynucleotides are directed may include, for example, connexins 26, 30, 30.3, 31.1, 32, 36, 37, 40, 40.1, 45, and 46.6. Suitable exemplary polynucleotides (and ODNs) directed to various connexins are set forth in Table 1.

Individual antisense polynucleotides may be specific to a particular connexin, or may target 1, 2, 3 or more different connexins. Specific polynucleotides will generally target sequences in the connexin gene or mRNA which are not conserved between connexins, whereas non-specific polynucleotides will target conserved sequences for various connexins.

The polynucleotides for use in the invention may suitably be unmodified phosphodiester oligomers. Such oligodeoxynucleotides may vary in length. A 30 mer polynucleotide has been found to be particularly suitable.

Many aspects of the invention are described with reference to oligodeoxynucleotides. However it is understood that other suitable polynucleotides (such as RNA polynucleotides) may be used in these aspects.

The antisense polynucleotides may be chemically modified. This may enhance their resistance to nucleases and may enhance their ability to enter cells. For example, phosphorothioate oligonucleotides may be used. Other deoxynucleotide analogs include methylphosphonates, phosphoramidates, phosphorodithioates, N3′P5′-phosphoramidates and oligoribonucleotide phosphorothioates and their 2′-O-alkyl analogs and 2′-O-methylribonucleotide methylphosphonates. Alternatively mixed backbone oligonucleotides (“MBOs”) may be used. MBOs contain segments of phosphothioate oligodeoxynucleotides and appropriately placed segments of modified oligodeoxy-or oligoribonucleotides. MBOs have segments of phosphorothioate linkages and other segments of other modified oligonucleotides, such as methylphosphonate, which is non-ionic, and very resistant to nucleases or 2′-O-alkyloligoribonucleotides. Methods of preparing modified backbone and mixed backbone oligonucleotides are known in the art.

The precise sequence of the antisense polynucleotide used in the invention will depend upon the target connexin protein. In one embodiment, suitable connexin antisense polynucleotides can include polynucleotides such as oligodeoxynucleotides selected from the following sequences set forth in Table 1:

TABLE 1

| | | |
|---|---|---|
| 5′ GTA ATT GCG GCA AGA AGA ATT GTT TCT GTC 3′ | (connexin 43) | (SEQ. ID. NO: 1) |
| 5′ GTA ATT GCG GCA GGA GGA ATT GTT TCT GTC 3′ | (connexin 43) | (SEQ. ID. NO: 2) |
| 5′ GGC AAG AGA CAC CAA AGA CAC TAC CAG CAT 3′ | (connexin 43) | (SEQ. ID. NO: 3) |
| 5′ TCC TGA GCA ATA CCT AAC GAA CAA ATA 3′ | (connexin 26) | (SEQ. ID. NO: 4) |
| 5′ CAT CTC CTT GGT GCT CAA CC 3′ | (connexin 37) | (SEQ. ID. NO: 5) |
| 5′ CTG AAG TCG ACT TGG CTT GG 3′ | (connexin 37) | (SEQ. ID. NO: 6) |
| 5′ CTC AGA TAG TGG CCA GAA TGC 3′ | (connexin 30) | (SEQ. ID. NO: 7) |
| 5′ TTG TCC AGG TGA CTC CAA GG 3′ | (connexin 30) | (SEQ. ID. NO: 8) |
| 5′ CGT CCG AGC CCA GAA AGA TGA GGT C 3′ | (connexin 31.1) | (SEQ. ID. NO: 9) |
| 5′ AGA GGC GCA CGT GAG ACA C 3′ | (connexin 31.1) | (SEQ. ID. NO: 10) |
| 5′ TGA AGA CAA TGA AGA TGT T 3′ | (connexin 31.1) | (SEQ. ID. NO: 11) |
| 5′ TTT CTT TTC TAT GTG CTG TTG GTG A 3′ | (connexin 32) | (SEQ. ID. NO: 12) |

Suitable polynucleotides for the preparation of the combined polynucleotide compositions described herein include for example, polynucleotides to connexin 43 and polynucleotides for connexins 26, 30, 31.1, 32 and 37 as described in Table 1 above.

Although the precise sequence of the antisense polynucleotide used in the invention will depend upon the target connexin protein, for connexin 43, antisense polynucleotides having the following sequences have been found to be particularly suitable:

(SEQ. ID. NO: 1)
GTA ATT GCG GCA AGA AGA ATT GTT TCT GTC;
(SEQ. ID. NO: 2)
GTA ATT GCG GCA GGA GGA ATT GTT TCT GTC;
and
(SEQ. ID. NO: 3)
GGC AAG AGA CAC CAA AGA CAC TAC CAG CAT.

For example, suitable antisense polynucleotides for connexins 26, 31.1 and 32 have the following sequences:

(SEQ. ID. NO: 4)
5′ TCC TGA GCA ATA CCT AAC GAA CAA ATA;
(connexin 26)
(SEQ. ID. NO: 9)
5′ CGT CCG AGC CCA GAA AGA TGA GGT C;
(connexin 31.1)
and
(SEQ. ID. NO: 12)
5′ TTT CTT TTC TAT GTG CTG TTG GTG A.
(connexin 32)

Other connexin antisense polynucleotide sequences useful according to the methods of the present invention include:

(SEQ. ID. NO: 5)
5′ CAT CTC CTT GGT GCT CAA CC 3′;
(connexin 37)
(SEQ. ID. NO: 6)
5′ CTG AAG TCG ACT TGG CTT GG 3′;
(connexin 37)
(SEQ. ID. NO: 7)
5′ CTC AGA TAG TGG CCA GAA TGC 3′;
(connexin 30)
(SEQ. ID. NO: 8)
5′ TTG TCC AGG TGA CTC CAA GG 3′;
(connexin 30)
(SEQ. ID. NO: 10)
5′ AGA GGC GCA CGT GAG ACA C 3′;
(connexin 31.1)
and
(SEQ. ID. NO: 11)
5′ TGA AGA CAA TGA AGA TGT T 3′.
(connexin 31.1)

Polynucleotides, including ODN's, directed to connexin proteins can be selected in terms of their nucleotide sequence by any convenient, and conventional, approach. For example, the computer programs MacVector and OligoTech (from Oligos etc. Eugene, Oreg., USA) can be used. Once selected, the ODN's can be synthesized using a DNA synthesizer.

### Polynucleotide Homologues

Anti-connexin polynucleotides also include polynucleotide homologues. Homology and homologues are discussed herein (for example, the polynucleotide may be a homologue of a complement to a sequence in connexin mRNA). Such a polynucleotide typically has at least about 70% homology, preferably at least about 80%, at least about 90%, at least about 95%, at least about 97% or at least about 99% homology with the relevant sequence, for example over a region of at least about 15, at least about 20, at least about 40, at least about 100 more contiguous nucleotides (of the homologous sequence).

Homology may be calculated based on any method in the art. For example the UWGCG Package provides the BESTFIT program, which can be used to calculate homology (for example used on its default settings) (Devereux et al. (1984) Nucleic Acids Research 12, p 387-395). The PILEUP and BLAST algorithms can be used to calculate homology or line up sequences (typically on their default settings), for example as described in Altschul S. F. (1993) J Mol Evol 36: 290-300; Altschul, S, F et al (1990) J Mol Biol 215: 403-10.

Software for performing BLAST analyses is publicly available through the National Center for Biotechnology Information (http://www.ncbi.nlm.nih.gov/). This algorithm involves first identifying high scoring sequence pair (HSPs) by identifying short words of length W in the query sequence that either match or satisfy some positive-valued threshold score T when aligned with a word of the same length in a database sequence. T is referred to as the neighbourhood word score threshold (Altschul et al, supra). These initial neighbourhood word hits act as seeds for initiating searches to find HSPs containing them. The word hits are extended in both directions along each sequence for as far as the cumulative alignment score can be increased. Extensions for the word hits in each direction are halted when: the cumulative alignment score falls off by the quantity X from its maximum achieved value; the cumulative score goes to zero or below, due to the accumulation of one or more negative-scoring residue alignments; or the end of either sequence is reached.

The BLAST algorithm parameters W, T and X determine the sensitivity and speed of the alignment. The BLAST program uses as defaults a word length (W), the BLOSUM62 scoring matrix (see Henikoff and Henikoff (1992) Proc. Natl. Acad. Sci. USA 89: 10915-10919) alignments (B) of 50, expectation (E) of 10, M=5, N=4, and a comparison of both strands.

The BLAST algorithm performs a statistical analysis of the similarity between two sequences; see e.g., Karlin and Altschul (1993) Proc. Natl. Acad. Sci. USA 90: 5873-5787. One measure of similarity provided by the BLAST algorithm is the smallest sum probability (P(N)), which provides an indication of the probability by which a match between two nucleotide or amino acid sequences would occur by chance. For example, a sequence is considered similar to another sequence if the smallest sum probability in comparison of the first sequence to a second sequence is less than about 1, preferably less than about 0.1, more preferably less than about 0.01, and most preferably less than about 0.001.

The homologous sequence typically differs from the relevant sequence by at least about (or by no more than about) 2, 5, 10, 15, 20 more mutations (which may be substitutions, deletions or insertions). These mutations may be measured across any of the regions mentioned above in relation to calculating homology.

The homologous sequence typically hybridizes selectively to the original sequence at a level significantly above background. Selective hybridization is typically achieved using conditions of medium to high stringency (for example 0.03M sodium chloride and 0.03M sodium citrate at from about 50° C. to about 60° C.). However, such hybridization may be carried out under any suitable conditions known in the art (see Sambrook et al. (1989), Molecular Cloning: A Laboratory Manual). For example, if high stringency is required, suitable conditions include 0.2×SSC at 60° C. If lower stringency is required, suitable conditions include 2×SSC at 60° C.

### Therapeutic Agents

Therapeutic agents include pharmaceutically acceptable agents useful in the treatment of wounds or the promotion of wound-healing, whether currently existing and known or later developed. Therapeutic agents include, for example, anti-infectives, anesthetics, analgesics, antibiotics, narcotics, and steroidal and non-steroidal anti-inflammatory agents. Preferred therapeutic agents include topical steroid anti-inflammatory agents, antimicrobial agents, local and topical anesthetics, and topical opioids. In certain embodiments, one, two three, four, five or six therapeutic agents may be used in combination.

### Agents Useful for Wound Healing

As used herein, agents useful for wound healing include stimulators, enhancers or positive mediators of the wound healing cascade which 1) promote or accelerate the natural wound healing process or 2) reduce effects associated with improper or delayed wound healing, which effects include, for example, adverse inflammation, epithelialization, angiogenesis and matrix deposition, and scarring and fibrosis.

Positive mediators, enhancers and stimulators include for example, an agent which may stimulate, enhance, facilitate, or accelerate (i.e., agonize) the quantity, quality or efficacy of wound healing or the active wound healing process, or a wound healing-associated growth factor or cytokine at a wound site, or the activation of a wound healing-associated growth factor or cytokine receptor. Such agents may include a wound healing-associated growth factor or cytokine or a partially modified form of a wound healing-associated growth factor or cytokine, for example. A partially modified form of wound healing-associated growth factor or cytokine may, for example, have a longer half-life than the natural wound healing-associated growth factor or cytokine. Alternatively, it may be an inhibitor of wound healing-associated growth factor or cytokine metabolism.

Agents useful for wound healing also include fibrogenesis modulating agents, which include, for example, any agent which can prevent and/or suppress, reduce or improve fibrogenic pathology. Exemplary fibrogenic modulators include, for example, direct or indirect regulators associated with the wound-associated inflammatory reaction, recruitment of neutrophils to the site of injury; activation and recruitment of macrophages and endothelial cells; recruitment and activation of lymphocytes and/or eosinophils via secretion of a number of cytokines/chemokines; release of cytotoxic mediators and fibrogenic cytokines; recruiting and activating cell proliferation, ECM synthesis and angiogenesis.

Partial modification of such an agent may be by way of addition, deletion or substitution of amino acid residues. A substitution may for example be a conserved substitution. Hence a partially modified molecule may be a homologue of the molecule from which it was derived. It may have at least about 40%, for example about 50, 60, 70, 80, 90 or 95%, homology with the molecule from which it is derived.

As used herein, agents useful for wound healing may include for example, wound-healing-promoting or scar-reducing agents for wound treatment modalities now known in the art or later-developed; exemplary factors, agents or modalities including natural or synthetic growth factors, cytokines, or modulators thereof to promote wound healing, wound healing promoting bioengineered matrix, dressings bandages, and the like. Suitable examples may include, but not limited to 1) topical or dressing and related therapies and debriding agents (such as, for example, Santyl® collagenase) and Iodosorb® (cadexomer iodine); 2) antimicrobial agents, including systemic or topical creams or gels, including, for example, silver-containing agents such as SAGs (silver antimicrobial gels), (CollaGUARD™, Innocoll, Inc) (purified type-I collagen protein based dressing), CollaGUARD Ag (a collagen-based bioactive dressing impregnated with silver for infected wounds or wounds at risk of infection), DermaSIL™ (a collagen-synthetic foam composite dressing for deep and heavily exuding wounds); 3) cell therapy or bioengineered skin, skin substitutes, and skin equivalents, including, for example, Dermograft (3-dimensional matrix cultivation of human fibroblasts that secrete cytokines and growth factors), Apligraf® (human keratinocytes and fibroblasts), Graftskin® (bilayer of epidermal cells and fibroblasts that is histologically similar to normal skin and produces growth factors similar to those produced by normal skin), TransCyte (a Human Fibroblast Derived Temporary Skin Substitute) and Oasis® (an active biomaterial that comprises both growth factors and extracellular matrix components such as collagen, proteoglycans, and glycosaminoglycans); 4) cytokines, growth factors or hormones (both natural and synthetic) introduced to the wound to promote wound healing, including, for example, NGF, NT3, BDGF, integrins, plasmin, semaphoring, blood-derived growth factor, keratinocyte growth factor, tissue growth factor, TGF-alpha, TGF-beta, PDGF (one or more of the three subtypes may be used: AA, AB, and B), PDGF-BB, TGF-beta 3, factors, that modulate the relative levels of TGFβ3, TGFβ1, and TGFβ2 (e.g., Mannose-6-phosphate), sex steroids, including for example, estrogen, estradiol, or an oestrogen receptor agonist selected from the group consisting of ethinyloestradiol, dienoestrol, mestranol, oestradiol, oestriol, a conjugated oestrogen, piperazine oestrone sulphate, stilboestrol, fosfesterol tetrasodium, polyestradiol phosphate, tibolone, a phytoestrogen, 17-beta-estradiol; thymic hormones such as Thymosin-beta-4, EGF, HB-EGF, fibroblast growth factors (e.g., FGF1, FGF2, FGF7), keratinocyte growth factor, TNF, interleukins family of inflammatory response modulators such as, for example, IL-10, IL-1, IL-2, IL-6, IL-8, and IL-10 and modulators thereof; INFs (INF-alpha, -beta, and -delta); stimulators of activin or inhibin, and inhibitors of interferon gamma prostaglandin E2 (PGE2) and of mediators of the adenosine 3′,5′-cyclic monophosphate (cAMP) pathway; adenosine A1 agonist, adenosine A2 agonist or 5) other agents useful for wound healing, including, for example, both natural or synthetic homologues, agonist and antagonist of VEGF, VEGFA, IGF; IGF-1, proinflammatory cytokines, GM-CSF, and leptins and 6) IGF-1 and KGF cDNA, autologous platelet gel, hypochlorous acid (Sterilox® lipoic acid, nitric oxide synthase3, matrix metalloproteinase 9 (MMP-9), CCT-ETA, alphavbeta6 integrin, growth factor-primed fibroblasts and Decorin, silver containing wound dressings, Xenaderm™, papain wound debriding agents, lactoferrin, substance P, collagen, and silver-ORC, placental alkaline phosphatase or placental growth factor, modulators of hedgehog signaling, modulators of cholesterol synthesis pathway, and APC (Activated Protein C), keratinocyte growth factor, TNF, Thromboxane A2, NGF, BMP bone morphogenetic protein, CTGF (connective tissue growth factor), wound healing chemokines, decorin, modulators of lactate induced neovascularization, cod liver oil, placental alkaline phosphatase or placental growth factor, and thymosin beta 4. In certain embodiments, one, two three, four, five or six agents useful for wound healing may be used in combination.

It is to be understood that the agents useful for wound healing (including for example, growth factors and cytokines) above encompass all naturally occurring polymorphs (for example, polymorphs of the growth factors or cytokines). Also, functional fragments, chimeric proteins comprising one of said agents useful for wound healing or a functional fragment thereof, homologues obtained by analogous substitution of one or more amino acids of the agent useful for wound healing, and species homologues are encompassed. It is contemplated that one or more agents useful for wound healing may be a product of recombinant DNA technology, and one or more agents useful for wound healing may be a product of transgenic technology. For example, platelet derived growth factor may be provided in the form of a recombinant PDGF or a gene therapy vector comprising a coding sequence for PDGF.

A fragment or partially modified form thereof refers to a fragment or partially modified form of the agent useful for wound healing which retains the biological or wound healing functionality of the factor, although it may of course have additional functionality. Partial modification may, for example, be by way of addition, deletion or substitution of amino acid residues. For example, a substitution may be a conserved substitution. Hence the partially modified molecules may be homologues of the agent useful for wound healing. They may, for example, have at least about 40% homology with said factor. They may for example have at least about 50, 60, 70, 80, 90 or 95% homology with said factor. For example, in certain embodiments, IL-10 or a fragment or a partially modified form thereof may be administered at a concentration of between about 1 μM and about 10 μM. It may be administered at a concentration of between about 2.5 μM and about 5 μM. In certain other embodiments, IL-10 or a fragment or a partially modified form thereof may be administered immediately prior to wound healing, but may be effective if administered within about 7 days of wounding. It could be administered on at least two occasions.

### Anti-Microtubule Agents

Exemplary anti-microtubule agents include, for example, diterpenoids (e.g. paclitaxel, docetaxel, and derivatives or analogues thereof) and vinca alkaloids (e.g. vinblastine, vincristine, and vinorelbine); platinum coordination complexes (e.g. cisplatin and carboplatin).

### Dosage Forms and Formulations and Administration

The agents of the invention of the may be administered to a subject in need of treatment, such as a subject with, or at risk for, any of the diseases, disorders or conditions mentioned herein. The condition of the subject can thus be improved. The anti-connexin polynucleotide may be used in the treatment of the subject's body by therapy. They may be used in the manufacture of a medicament to treat any of the diseases, disorders or conditions mentioned herein.

The anti-connexin polynucleotide may be present in a substantially isolated faun. It will be understood that the product may be mixed with carriers or diluents which will not interfere with the intended purpose of the product and still be regarded as substantially isolated. A product of the invention may also be in a substantially purified form, in which case it will generally comprise about 80%, 85%, or 90%, including, for example, at least about 95%, at least about 98% or at least about 99% of the polynucleotide or dry mass of the preparation.

Depending on the intended route of administration, the pharmaceutical products, pharmaceutical compositions, combined preparations and medicaments of the invention may, for example, take the form of solutions, suspensions, instillations, sprays, salves, creams, gels, foams, ointments, emulsions, lotions, paints, sustained release formulations, or powders, and typically contain about 0.01% to about 1% of active ingredient(s), about 1%-50% or active ingredient(s), about 2%-60% of active ingredient(s), about 2%-70% of active ingredient(s), or up to about 90% of active ingredient(s). Other suitable formulations include pluronic gel-based formulations, carboxymethylcellulose (CMC)-based formulations, and hyroxypropylmethylcellulose (HPMC)-based formulations. Other useful formulations include slow or delayed release preparations.

Gels or jellies may be produced using a suitable gelling agent including, but not limited to, gelatin, tragacanth, or a cellulose derivative and may include glycerol as a humectant, emollient, and preservative. Ointments are semi-solid preparations that consist of the active ingredient incorporated into a fatty, waxy, or synthetic base. Examples of suitable creams include, but are not limited to, water-in-oil and oil-in-water emulsions. Water-in-oil creams may be formulated by using a suitable emulsifying agent with properties similar, but not limited, to those of the fatty alcohols such as cetyl alcohol or cetostearyl alcohol and to emulsifying wax. Oil-in-water creams may be formulated using an emulsifying agent such as cetomacrogol emulsifying wax. Suitable properties include the ability to modify the viscosity of the emulsion and both physical and chemical stability over a wide range of pH. The water soluble or miscible cream base may contain a preservative system and may also be buffered to maintain an acceptable physiological pH.

Foam preparations may be formulated to be delivered from a pressurized aerosol canister, via a suitable applicator, using inert propellants. Suitable excipients for the formulation of the foam base include, but are not limited to, propylene glycol, emulsifying wax, cetyl alcohol, and glyceryl stearate. Potential preservatives include methylparaben and propylparaben.

Preferably the agents of the invention are combined with a pharmaceutically acceptable carrier or diluent to produce a pharmaceutical composition. Suitable carriers and diluents include isotonic saline solutions, for example phosphate-buffered saline. Suitable diluents and excipients also include, for example, water, saline, dextrose, glycerol, or the like, and combinations thereof. In addition, if desired substances such as wetting or emulsifying agents, stabilizing or ph buffering agents may also be present.

The term “pharmaceutically acceptable carrier” refers to any pharmaceutical carrier that does not itself induce the production of antibodies harmful to the individual receiving the composition, and which can be administered without undue toxicity. Suitable carriers can be large, slowly metabolized macromolecules such as proteins, polysaccharides, polylactic acids, polyglycolic acids, polymeric amino acids, and amino acid copolymers.

Pharmaceutically acceptable salts can also be present, e.g., mineral acid salts such as hydrochlorides, hydrobromides, phosphates, sulfates, and the like; and the salts of organic acids such as acetates, propionates, malonates, benzoates, and the like.

Suitable carrier materials include any carrier or vehicle commonly used as a base for creams, lotions, sprays, foams, gels, emulsions, lotions or paints for topical administration. Examples include emulsifying agents, inert carriers including hydrocarbon bases, emulsifying bases, non-toxic solvents or water-soluble bases. Particularly suitable examples include pluronics, HPMC, CMC and other cellulose-based ingredients, lanolin, hard paraffin, liquid paraffin, soft yellow paraffin or soft white paraffin, white beeswax, yellow beeswax, cetostearyl alcohol, cetyl alcohol, dimethicones, emulsifying waxes, isopropyl myristate, microcrystalline wax, oleyl alcohol and stearyl alcohol.

Preferably, the pharmaceutically acceptable carrier or vehicle is a gel, suitably a nonionic polyoxyethylene-polyoxypropylene copolymer gel, for example, a Pluronic gel, preferably Pluronic F-127 (BASF Corp.). This gel is particularly preferred as it is a liquid at low temperatures but rapidly sets at physiological temperatures, which confines the release of the agent to the site of application or immediately adjacent that site.

An auxiliary agent such as casein, gelatin, albumin, glue, sodium alginate, carboxymethylcellulose, methylcellulose, hydroxyethylcellulose or polyvinyl alcohol may also be included in the formulation of the invention.

Other suitable formulations include pluronic gel-based formulations, carboxymethylcellulose (CMC)-based formulations, and hydroxypropylmethylcellulose (HPMC)-based formulations. The composition may be formulated for any desired form of delivery, including topical, instillation, parenteral, intramuscular, subcutaneous, or transdermal administration. Other useful formulations include slow or delayed release preparations.

The formulation which is administered may contain transfection agents. Examples of such agents include cationic agents (for example calcium phosphate and DEAE-dextran) and lipofectants (for example lipofectam™ and transfectam™), and surfactants.

In one embodiment, the formulation further includes a surfactant to assist with polynucleotide cell penetration or the formulation may contain any suitable loading agent. Any suitable non-toxic surfactant may be included, such as DMSO. Alternatively a transdermal penetration agent such as urea may be included.

Optionally, the anti-connexin polynucleotide may be formulated with one or more therapeutic agents, agents useful for wound healing, and/or anti-fibrotic agents. In certain embodiments, one, two three, four, five or six therapeutic agents may be used in combination. In certain embodiments, one, two three, four, five or six agents useful for wound healing may be used in combination.

In one aspect, the one or more anti-connexin polynucleotides, either alone or in combination with one or more therapeutic agents and/or agents useful in wound healing are provided in the form of a wound dressing or matrix. In certain embodiments, the one or more anti-connexin polynucleotides (with or without one or more therapeutic agents or agents useful in wound healing) are provided in the form of a liquid, semi solid or solid composition for application directly, or the composition is applied to the surface of or incorporated into, a solid contacting layer such as a dressing gauze or matrix. The wound dressing composition may be provided for example, in the form of a fluid or a gel. The one or more anti-connexin polynucleotides (with or without one or more therapeutic agents or agents useful in wound healing) may be provided in combination with conventional pharmaceutical excipients for topical application. Suitable carriers include: Pluronic gels, Polaxamer gels, Hydrogels containing cellulose derivatives, including hydroxyethyl cellulose, hydroxymethyl cellulose, carboxymethyl cellulose, hydroxypropylmethyl cellulose and mixtures thereof; and hydrogels containing polyacrylic acid (Carbopols). Suitable carriers also include creams/ointments used for topical pharmaceutical preparations, e.g., creams based on cetomacrogol emulsifying ointment. The above carriers may include alginate (as a thickener or stimulant), preservatives such as benzyl alcohol, buffers to control pH such as disodium hydrogen phosphate/sodium dihydrogen phosphate, agents to adjust osmolarity such as sodium chloride, and stabilizers such as EDTA.

The effective dose for a given subject preferably lies within the dose that is therapeutically effective for at least 50% of the population, and that exhibits little or no toxicity at this level.

The effective dosage of each of the anti-connexin polynucleotides employed in the methods and compositions of the invention may vary depending on a number of factors including the particular anti-connexin polynucleotide employed, the mode of administration, the frequency of administration, the wound being treated, the severity of the wound being treated, the route of administration, the needs of a patient sub-population to be treated or the needs of the individual patient which different needs can be due to age, sex, body weight, relevant medical wound specific to the patient.

A suitable dose may be from about 0.001 to about 1 mg/kg body weight such as about 0.01 to about 0.4 mg/kg body weight. A suitable dose may however be from about 0.001 to about 0.1 mg/kg body weight such as about 0.01 to about 0.050 mg/kg body weight. Doses from about 1 to 100, 100-200, 200-300, 300-400, and 400-500 micrograms or more and up to about 500-1000 micrograms are appropriate. As noted herein, repeat applications are contemplated. Repeat applications are typically applied about once per week, or when healing may appear to be stalled or slowing.

Still other dosage levels between about 1 nanogram (ng)/kg and about 1 mg/kg body weight per day of each of the agents described herein. In certain embodiments, the dosage of each of the subject compounds will generally be in the range of about 1 ng to about 1 microgram per kg body weight, about 1 ng to about 0.1 microgram per kg body weight, about 1 ng to about 10 ng per kg body weight, about 10 ng to about 0.1 microgram per kg body weight, about 0.1 microgram to about 1 microgram per kg body weight, about 20 ng to about 100 ng per kg body weight, about 0.001 mg to about 100 mg per kg body weight, about 0.01 mg to about 10 mg per kg body weight, or about 0.1 mg to about 1 mg per kg body weight. In certain embodiments, the dosage of each of the subject compounds will generally be in the range of about 0.001 mg to about 0.01 mg per kg body weight, about 0.01 mg to about 0.1 mg per kg body weight, about 0.1 mg to about 1 mg per kg body weight, or about 1 mg per kg body weight. If more than one anti-connexin polynucleotide is used, the dosage of each anti-connexin polynucleotide need not be in the same range as the other. For example, the dosage of one anti-connexin polynucleotide may be between about 0.01 mg to about 1 mg per kg body weight, and the dosage of another anti-connexin polynucleotide may be between about 0.1 mg to about 1 mg per kg body weight. As noted herein, repeat applications are contemplated. Repeat applications are typically applied about once per week, or when wound-healing may appear to be stalled or slowing.

Other useful doses range from about 1 to about 10 micrograms per square centimeter of the size of the wound or the area to be treated. Certain doses will be about 1-2, about 1-5, about 2-4, about 5-7, and about 8-10 micrograms per square centimeter of the size of the wound or the area to be treated. Other useful doses are greater than about 10 micrograms per square centimeter of the size of the wound or the area to be treated, including about 15 micrograms per square centimeter of the size of the wound or the area to be treated, about 20 micrograms per square centimeter of the size of the wound or the area to be treated, about 25 micrograms per square centimeter of the size of the wound or the area to be treated, about 30 micrograms per square centimeter of the size of the wound or the area to be treated, about 35 micrograms per square centimeter of the size of the wound or the area to be treated, about 40 micrograms per square centimeter of the size of the wound or the area to be treated, about 50 micrograms per square centimeter of the size of the wound or the area to be treated, and about 100 micrograms per square centimeter of the size of the wound or the area to be treated. Other useful doses are about 150 micrograms per square centimeter of the size of the wound or the area to be treated, about 200 micrograms per square centimeter of the size of the wound or the area to be treated, about 250 micrograms per square centimeter of wound size, or about 500 micrograms per square centimeter of the size of the wound or the area to be treated. As noted herein, repeat applications are contemplated. Repeat applications are typically applied about once per week, or when wound-healing may appear to be stalled or slowing.

For example, in certain embodiments, the anti-connexin polynucleotide composition may be applied at about 0.01 micromolar (μM) or 0.05 μM to about 200 μM final concentration at the treatment site and/or adjacent to the treatment site. Preferably, the antisense polynucleotide composition is applied at about 0.05 μM to about 100 μM final concentration, more preferably, the anti-connexin polynucleotide composition is applied at about 1.0 μM to about 50 μM final concentration, and more preferably, the anti-connexin polynucleotide composition is applied at about 5-10 μM to about 30-50 μM final concentration. Additionally, the anti-connexin polynucleotide composition is applied at about 8 μM to about 20 μM final concentration, and alternatively the anti-connexin polynucleotide composition is applied at about 10 μM to about 20 μM final concentration, or at about 10 to about 15 μM final concentration. In certain other embodiments, the anti-connexin polynucleotide is applied at about 10 μM final concentration. In yet another embodiment, the anti-connexin polynucleotide composition is applied at about 1-15 μM final concentration. The dose at which an anti-connexin agent is administered to a patient will depend upon a variety of factors such as the age, weight and general condition of the patient, the condition that is being treated, and the particular anti-connexin agent that is being administered.

A suitable therapeutically effective dose of an anti-connexin agent may be from about 0.001 to about 1 mg/kg body weight such as about 0.01 to about 0.4 mg/kg body weight. A suitable dose may however be from about 0.001 to about 0.1 mg/kg body weight such as about 0.01 to about 0.050 mg/kg body weight.

Therapeutically effective doses of anti-connexin agents from about 1 to 100, 100-200, 100- or 200-300, 100- or 200- or 300-400, and 100- or 200- or 300- or 400-500 micrograms are appropriate. Doses from about 1-1000 micrograms are also appropriate. Doses up to 2 milligrams may also be used. Doses are adjusted appropriately when the anti-connexin agent or agents are provided in the form of a dressing, typically upward to maintain the desired total dose administration.

Alternatively, in the case of anti-connexin oligonucleotides, the dosage of each of the agents in the compositions may be determined by reference to the composition's concentration relative to the size, length, depth, area or volume of the area to which it will be applied. For example, in certain topical applications, dosing of the pharmaceutical compositions may be calculated based on mass (e.g. grams) of or the concentration in a pharmaceutical composition (e.g. μg/μl) per length, depth, area, or volume of the area of application. Useful doses range from about 1 to about 10 micrograms per square centimeter of wound size. Certain doses will be about 1-2, about 1-5, about 2-4, about 5-7, and about 8-10 micrograms per square centimeter of wound size. Other useful doses are greater than about 10 micrograms per square centimeter of wound size, including at least about 15 micrograms per square centimeter of wound size, at least about 20 micrograms per square centimeter of wound size, at least about 25 micrograms per square centimeter of wound size, about 30 micrograms per square centimeter of wound size, at least about 35 micrograms per square centimeter of wound size, at least about 40 micrograms per square centimeter of wound size, at least about 50 micrograms per square centimeter of wound size, and at least about 100 to at least about 150 micrograms per square centimeter of wound size. Other doses include about 150-200 micrograms per square centimeter, about 200-250 micrograms per square centimeter, about 250-300 micrograms per square centimeter, about 300-350 micrograms per square centimeter, about 350-400 micrograms per square centimeter, and about 400-500 micrograms per square centimeter.

In certain embodiments, the anti-connexin polynucleotide composition may be applied at about 0.01 micromolar (μM) or 0.05 μM to about 200 μM, or up to 300 μM or up to 1000 μM or up to 2000 μM or up to 3200 μM or more final concentration at the treatment site and/or adjacent to the treatment site, and any doses and dose ranges within these dose numbers. Preferably, the antisense polynucleotide composition is applied at about 0.05 μM to about 100 μM final concentration, more preferably, the anti-connexin polynucleotide composition is applied at about 1.0 μM to about 50 μM final concentration, and more preferably, the anti-connexin polynucleotide composition is applied at about 5-10 μM to about 30-50 μM final concentration. Additionally, the combined anti-connexin polynucleotide composition is applied at about 8 μM to about 20 μM final concentration, and alternatively the anti-connexin polynucleotide composition is applied at about 10 μM to about 20 μM final concentration, or at about 10 to about 15 μM final concentration. In certain other embodiments, the anti-connexin polynucleotide is applied at about 10 μM final concentration. In yet another embodiment, the anti-connexin polynucleotide composition is applied at about 1-15 μM final concentration. In other embodiments, the anti-connexin polynucleotide is applied at about a 20 μM, 30 μM, 40 μM, 50 μM, 60 μM, 70 μM, 80 μM, 90 μM, 100 μM., 10-200 μM, 200-300 μM, 300-400 μM, 400-500 μM, 500-600 μM, 600-700 μM, 700-800 μM, 800-900 μM, 900-1000 or 1000-1500 μM, or 1500 μM-2000 μM or 2000 μM-3000 μM or greater.

Anti-connexin polynucleotide dose amounts include, for example, about 0.1-1, 1-2,2-3, 3-4, or 4-5 micrograms (μg), from about 5 to about 10 μg, from about 10 to about 15 μg, from about 15 to about 20 μg, from about 20 to about 30 μg, from about 30 to about 40 μg, from about 40 to about 50 μg, from about 50 to about 75 μg, from about 75 to about 100 μg, from about 100 μg to about 250 μg, and from 250 μg to about 500 μg. Dose amounts from 0.5 to about 1.0 milligrams or more or also provided, as noted above. Dose volumes will depend on the size of the site to be treated, and may range, for example, from about 25-100 μL to about 100-200 μL, from about 200-500 μL to about 500-1000 μL. Milliliter doses are also appropriate for larger treatment sites. As noted herein, repeat applications are contemplated. Repeat applications are typically applied about once per week, or when wound-healing may appear to be stalled or slowing.

Conveniently, the anti-connexin polynucleotide is administered in a sufficient amount to downregulate expression of a connexin protein, or modulate gap junction formation for at least about 0.5 to 1 hour, at least about 1-2 hours, at least about 2-4 hours, at least about 4-6 hours, at least about 6-8 hours, at least about 8-10 hours, at least about 12 hours, or at least about 24 hours post-administration.

The dosage of each of the anti-connexin polynucleotides in the compositions and methods of the subject invention may also be determined by reference to the concentration of the composition relative to the size, length, depth, area or volume of the area to which it will be applied. For example, in certain topical and other applications, e.g., instillation, dosing of the pharmaceutical compositions may be calculated based on mass (e.g. micrograms) of or the concentration in a pharmaceutical composition (e.g. μg/μl) per length, depth, area, or volume of the area of application.

The initial and any subsequent dosages administered will depend upon factors noted herein. Depending on the oligonucleotide, the dosage and protocol for administration will vary, and the dosage will also depend on the method of administration selected, for example, local or topical administration.

The doses may be administered in single or divided applications. The doses may be administered once, or application may be repeated. Typically, application will be repeated weekly until healing is promoted, or a repeat application may be made in the event that healing slows or is stalled. Doses may be applied 3-7 days apart, or more. Repeat applications may be made, for example, weekly, or bi-weekly, or monthly or in other frequency for example if and when wound healing slows or is stalled. For some indications, such as certain ocular uses, more frequent dosing, up to hourly may employed.

Agents useful for wound healing suitable for the preparation of the pharmaceutical compositions described herein may be prepared and administered using methods as known in the art (see, for example, U.S. Pat. Nos. 7,098,190, 6,319,907, 6,331,298, 6,387,364, 6,455,569, 6,566,339, 6,696,433, 6,855,505, 6,900,181, 7,052,684 and EP1100529 B1. The concentration of each anti-connexin polynucleotide and agent useful for wound healing need not be in the same range as the other. Other amounts will be known to those of skill in the art and readily determined. For example, suitable combination dosages and formulations in accordance with various aspects and embodiments as described herein may be administered according to the dosing regimen as described in U.S. Pat. No. 6,903,078 to Lewis entitled “Combination PDGF, KGF, IGF, and IGFBP for wound healing.”

The initial and any subsequent dosages administered will depend upon the patient's age, weight, condition, and the disease, wound, disorder or biological condition being treated. Depending on the agent useful for wound healing, the dosage and protocol for administration will vary, and the dosage will also depend on the method of administration selected, for example, local or systemic administration.

The agent useful for wound healing may be applied internally or externally, and may be directed towards any tissue exhibiting a fibrotic lesion or area, or at risk thereof. For topical administration of IGF, for example, a zinc oxide formulation can be applied, which induces the local production of IGF, as described in Tarnow et al, Scand J. Plast Reconstr Hand Surg. 28: 255-259 (1994). An effective dose of PDGF has been reported to be 5 ng/mm² or higher when applied topically as described in U.S. Pat. No. 4,861,757, and at least 1 ng/ml local concentration of an isoform of PDGF (for example, PDGF-AA, PDGF-BB, or PDGF-AB), up to about 30 ng/ml local concentration applied to a population of fibroblasts as described in Lepisto et al., Biochem Biophys Res. Comm 209: 393-399 (1995). PDGF can be administered in a carboxymethylcellulose gel formulation at concentrations of about 10 μg/gm to about 500 μg/gm of gel, about 20 μg/gm to about 200 μg/gm, and about 30 μg/gm to about 100 g g/gm of gel, optimally about 100 μg/gm of gel. Efficacy of PDGF has been achieved within the range of about 3 μg/ml solution to about 300 μg/ml of solution administered.

About 50 μl of KGF of a concentration of about 5 μg/ml may be effective for wound healing by topical application to epithelial tissue as described in Sotozono et al, Invest. Opthal. Vis. Science 36: 1524-29 (1995). As described in U.S. Pat. No. 4,861,757, an effective amount of IGF when co-administered with PDGF is in the range of at least 2.5 ng/mm² to about 5 ng/mm², with a ratio of PDGF to IGF in the range of about 1:10 to about 25:1 weight to weight, with the most effective ratios being PDGF to IGF of about 1:1 to about 2:1 weight to weight. IGFBP administered in combination with IGF has been shown to increase wound healing at dose levels of about 5 μg of IGF with about 1.5 μg of phosphorylated IGFBP in a molar ration of about 11:1 IGF:IGFBP, as described in Jyung et al, Surgery 115:233-239 (1994).

For administration of polypeptide therapeutics, for example, PDGF, KGF, IGF and IGFBP polypeptides, the dosage can be in the range of about 5 μg to about 50 μg/kg of tissue to which the application is directed, also about 50 μg to about 5 mg/kg, also about 100 μg to about 500 μg/kg of tissue, and about 200 to about 250 μg/kg. For polynucleotide therapeutics, for example in a gene therapy administration protocol, depending on the expression strength the polynucleotide in the patient, for tissue targeted administration, vectors containing expressible constructs including PDGF, KGF, IGF, and IGFBP coding sequences can be administered in a range of about 100 ng to about 200 mg of DNA for local administration in a gene therapy protocol, also about 500 ng to about 50 mg, also about 1 μg to about 2 mg of DNA, about 5 μg of DNA to about 500 μg of DNA, and about 20 μg to about 100 μg during a local administration in a gene therapy protocol, and about 250 μg, per injection or administration. Factors such as method of action and efficacy of transformation and expression are therefore considerations that will effect the dosage required for ultimate efficacy for administration of DNA therapeutics. Where greater expression is desired, over a larger area of tissue, larger amounts of DNA or the same amounts re-administered in a successive protocol of administrations, or several administrations to different adjacent or close tissue portions of for example, a wound site may be required to effect a positive therapeutic outcome.

Therapeutic agents suitable for the preparation of the pharmaceutical compositions described herein may be formulated and administered using methods as known in the art. The initial and any subsequent dosages administered will depend upon the patient's age, weight, condition, and the disease, wound, disorder or biological condition being treated. Depending on the therapeutic, the dosage and protocol for administration will vary, and the dosage will also depend on the method of administration selected, for example, local or systemic administration.

As noted herein, the doses of either an anti-connexin polynucleotides or another agent administered in combination can be adjusted down from the doses administered when given alone.

In another preferred embodiment, the combined use of one or more anti-connexin polynucleotides and one or more therapeutic agents and/or agents useful for wound healing reduces the effective dose of any such agent compared to the effective dose when said agent administered alone. In certain embodiments, the effective dose of the agent when used in combination with one or more anti-connexin polynucleotides is about 1/15 to about ½, about 1/10 to about ⅓, about ⅛ to about ⅙, about ⅕, about ¼, about ⅓ or about ½ the dose of the agent when used alone.

In another preferred embodiment, the combined use of one or more anti-connexin polynucleotides and one or more therapeutic agents and/or agents useful for wound healing reduces the frequency in which said agent is administered compared to the frequency when said agent is administered alone. Thus, these combinations allow the use of lower and/or fewer doses of each agent than previously required to achieve desired therapeutic goals.

The doses may be administered in single or divided applications. The doses may be administered once, or application may be repeated.

One or more anti-connexin polynucleotides, either alone or in combination with one or more therapeutic agents and/or one or more agents useful in wound healing, may be administered by the same or different routes. The various agents of the invention can be administered separately at different times during the course of therapy, or concurrently in divided or single combination forms.

Preferably one or more anti-connexin polynucleotides useful in the treatment of fibrosis are delivered by topical administration (peripherally or directly to a site), including but not limited to topical administration using solid supports (such as dressings and other matrices) and medicinal formulations (such as gels, mixtures, suspensions and ointments). In one embodiment, the solid support comprises a biocompatible membrane or insertion into a treatment site. In another embodiment, the solid support comprises a dressing or matrix. In one embodiment of the invention, the solid support composition may be a slow release solid support composition, in which the one or more anti-connexin polynucleotides useful for wound healing is dispersed in a slow release solid matrix such as a matrix of alginate, collagen, or a synthetic bioabsorbable polymer. Preferably, the solid support composition is sterile or low bio-burden. In one embodiment, a wash solution comprising one or more anti-connexin polynucleotides can be used.

In another embodiment, lavage solution containing about 1 to about 100 μg/cm² (preferably about 10 to about 50 μg/cm²) of an anti-connexin agent, would be used at the time of or immediately following injury or surgery. In all of the embodiments, other anti-connexin polynucleotides would be administered at equivalent doses adjusted for potency and tolerability of the polynucleotide.

The delivery of one or more anti-connexin polynucleotides (with or without one or more therapeutic agents or agents useful for wound healing) may occur over a period of time, in some instances for about 0.5 hours, 1-2 hours, about 2-4 hours, about 4-6 hours, about 6-8, or about 24 hours or longer, may be a particular advantage in more severe wounds. In some instances, cell loss may extend well beyond the site of a procedure to surrounding cells. Such loss may occur within 24 hours of the original procedure and is mediated by gap junction cell-cell communication. Administration of anti-connexin polynucleotide(s) will modulate communication between the cells and minimize additional cell loss or injury or consequences of injury.

While the delivery period will be dependent upon both the site at which the downregulation is to be induced and the therapeutic effect which is desired, continuous or slow-release delivery for about 0.5 hours, about 1-2 hours, about 2-4 hours, about 4-6 hours, about 6-8, or about 24 hours or longer is provided. In accordance with the present invention, this maybe achieved by inclusion of the anti-connexin polynucleotides (with or without one or more therapeutic agents or agents useful for wound healing) in a formulation together with a pharmaceutically acceptable carrier or vehicle, particularly in the form of a formulation for continuous or slow-release administration.

The routes of administration and dosages described herein are intended only as a guide since a skilled physician will determine the optimum route of administration and dosage for any particular patient.

Any of the methods of treating a subject having or suspected of having or a disease, disorder, or condition referenced or described herein may utilize the administration of any of the doses, dosage forms, formulations, and/or compositions herein described.

Therapeutic agents and anti-microtubule agents suitable for the preparation of the pharmaceutical compositions described herein may be formulated and administered using methods as known in the art. The initial and any subsequent dosages administered will depend upon the patient's age, weight, condition, and the disease, wound, disorder or biological condition being treated. Depending on the therapeutic, the dosage and protocol for administration will vary, and the dosage will also depend on the method of administration selected, for example, local or systemic administration.

As noted herein, the doses of either an anti-connexin polynucleotides or another agent administered in combination can be adjusted down from the doses administered when given alone.

The combined use of several agents may reduce the required dosage for any individual agent because the onset and duration of effect of the different agents may be complementary. In a preferred embodiment, the combined use of one or more anti-connexin polynucleotides and one or more therapeutic agents, agents useful for wound healing, and/or anti-microtubule agents has an additive, synergistic or super-additive effect.

In some cases, the combination of one or more anti-connexin polynucleotides and one or more therapeutic agents, one or more agents useful for wound healing, and/or one or more anti-microtubule agents have an additive effect. In other cases, the combination can have greater-than-additive effect. Such an effect is referred to herein as a “supra-additive” effect, and may be due to synergistic or potentiated interaction.

The term “supra-additive promotion of wound healing” refers to a mean wound healing produced by administration of a combination of an anti-connexin polynucleotide and one or more therapeutic agents, agents useful for wound healing and/or anti-microtubule agents, is statistically significantly higher than the sum of the wound healing produced by the individual administration of either any of the agents alone. Whether produced by combination administration of an anti-connexin polynucleotide and one or more therapeutic agents, agents useful for wound healing, and/or anti-microtubule agents is “statistically significantly higher” than the expected additive value of the individual compounds may be determined by a variety of statistical methods as described herein and/or known by one of ordinary skill in the art. The term “synergistic” refers to a type of supra-additive inhibition in which both the anti-connexin polynucleotide and one or more therapeutic agents, agents useful for wound healing and/or anti-microtubule agents individually have the ability to promote wound healing or reduce fibrosis and scarring. The term “potentiated” refers to type of supra-additive effect in which one of the anti-connexin polynucleotide or one or more therapeutic agents, agents useful for wound healing, and/or anti-microtubule agents individually has the increased ability to promote wound healing.

In general, potentiation may be assessed by determining whether the combination treatment produces a mean wound healing increase in a treatment group that is statistically significantly supra-additive when compared to the sum of the mean wound healing increases produced by the individual treatments in their treatment groups respectively. The mean wound healing increase may be calculated as the difference between control group and treatment group mean wound healing. The fractional increase in wound healing, “fraction affected” (Fa), may be calculated by dividing the treatment group mean wound healing increase by control group mean wound healing. Testing for statistically significant potentiation requires the calculation of Fa for each treatment group. The expected additive Fa for a combination treatment may be taken to be the sum of mean Fas from groups receiving either element of the combination. The Two-Tailed One-Sample T-Test, for example, may be used to evaluate how likely it is that the result obtained by the experiment is due to chance alone, as measured by thep-value. Ap-value of less than 0.05 is considered statistically significant, that is, not likely to be due to chance alone. Thus, Fa for the combination treatment group must be statistically significantly higher than the expected additive Fa for the single element treatment groups to deem the combination as resulting in a potentiated supra-additive effect.

Whether a synergistic effect results from a combination treatment may be evaluated by the median-effect/combination-index isobologram method (Chou, T., and Talalay, P. (1984) Ad. Enzyme Reg. 22:27-55). In this method, combination index (CI) values are calculated for different dose-effect levels based on parameters derived from median-effect plots of the anti-connexin polynucleotide alone, the one or more agents useful for wound healing alone, and the combination of the two at fixed molar ratios. CI values of & It; 1 indicate synergy, CI-1 indicates an additive effect, and CP1 indicates an antagonistic effect. This analysis may be performed using computer software tools, such as CalcuSyn, Windows Software for Dose Effect Analysis (Biosoft (D, Cambridge UK).

Any method known or later developed in the art for analyzing whether a supra-additive effect exists for a combination therapy is contemplated for use in screening for suitable anti-connexin polynucleotides for use in combination with one or more therapeutic agents, agents useful for wound healing and/or anti-microtubule agents.

In another preferred embodiment, the combined use of one or more anti-connexin polynucleotides and one or more therapeutic agents, agents useful for wound healing, and/or anti-microtubule agents reduces the effective dose of any such agent compared to the effective dose when said agent administered alone. In certain embodiments, the effective dose of the agent when used in combination with one or more anti-connexin polynucleotides is about 1/15 to about ½, about 1/10 to about ⅓, about ⅛ to about ⅙, about ⅕, about ¼, about ⅓ or about ½ the dose of the agent when used alone.

In another preferred embodiment, the combined use of one or more anti-connexin polynucleotides and one or more therapeutic agents, agents useful for wound healing, and/or anti-microtubule agents reduces the frequency in which said agent is administered compared to the frequency when said agent is administered alone. Thus, these combinations allow the use of lower and/or fewer doses of each agent than previously required to achieve desired therapeutic goals.

The doses may be administered in single or divided applications. The doses may be administered once, or application may be repeated.

One or more anti-connexin polynucleotides, either alone or in combination with one or more therapeutic agents, one or more agents useful in wound healing and/or one or more anti-microtubule agents, may be administered by the same or different routes. The various agents of the invention can be administered separately at different times during the course of therapy, or concurrently in divided or single combination forms.

Preferably one or more anti-connexin polynucleotides (with or with out one or more therapeutic agents, agents useful for wound healing and/or anti-microtubule agents) are delivered by topical administration (peripherally or directly to a site), including but not limited to topical administration using solid supports (such as dressings and other matrices) and medicinal formulations (such as gels, mixtures, suspensions and ointments). In one embodiment, the solid support comprises a biocompatible membrane or insertion into a treatment site. In another embodiment, the solid support comprises a dressing or matrix. In one embodiment of the invention, the solid support composition may be a slow release solid support composition, in which the one or more anti-connexin polynucleotides useful for wound healing is dispersed in a slow release solid matrix such as a matrix of alginate, collagen, or a synthetic bioabsorbable polymer. Preferably, the solid support composition is sterile or low bio-burden. In one embodiment, a wash solution comprising one or more anti-connexin polynucleotides can be used.

The anti-connexin agent (with or without one or more therapeutic agents, agents useful for wound healing and/or anti-microtubule agents) can be administered in any manner which achieves a desired result. Preferred methods include peritubular administration (either direct application at the time of surgery or with endoscopic, ultrasound, CT, MRI, or fluoroscopic guidance); “coating” the surgical implant; and placement of a drug-eluting polymeric implant at the surgical site. In a preferred embodiment, 0.5% to 20% anti-connexin polynucleotide by weight is loaded into a polymeric carrier (as described in the following examples) and applied to the peritubular (mesenteric) surface as a “paste”, “film”, or “wrap” which releases the drug over a period of time. During endoscopic procedures, the anti-connexin polymer preparation may be applied as a “spray”, via delivery ports in the endoscope, to the mesentery of the abdominal and pelvic organs manipulated during the operation. In a particularly preferred embodiment, the peritubular composition is about 0.1% to about 5% anti-connexin polynucleotide by weight. In another preferred embodiment, a polymeric coating containing about 0.1% to about 20% or more or an anti-connexin agent is applied to the surface of the surgical implant (e.g., breast implant, artificial joint, vascular graft, etc.). In yet another preferred embodiment, a polymeric implant containing about 0.01% to about 20% or more of an anti-connexin agent by weight is applied directly to the surgical site (e.g., directly into the sinus cavity, chest cavity, abdominal cavity, or at the operative site during neurosurgery).

In another embodiment, lavage fluid containing about 1 to about 100 μg/cm² (preferably about 10 to about 50 μg/cm²) of an anti-connexin agent, would be used at the time of or immediately following surgery and administered during surgery or intraperitoncally, by a physician. In all of the embodiments, other anti-connexin polynucleotides would be administered at equivalent doses adjusted for potency and tolerability of the polynucleotide.

The delivery of one or more anti-connexin polynucleotides (with or without one or more therapeutic agents, agents useful for wound healing, and/or anti-microtubule agents) may occur over a period of time, in some instances for about 0.5 hours, 1-2 hours, about 2-4 hours, about 4-6 hours, about 6-8, or about 24 hours or longer, may be a particular advantage in more severe wounds. In some instances, cell loss may extend well beyond the site of a procedure to surrounding cells. Such loss may occur within 24 hours of the original procedure and is mediated by gap junction cell-cell communication. Administration of anti-connexin polynucleotide(s) will modulate communication between the cells and minimize additional cell loss or injury or consequences of injury.

While the delivery period will be dependent upon both the site at which the downregulation is to be induced and the therapeutic effect which is desired, continuous or slow-release delivery for about 0.5 hours, about 1-2 hours, about 2-4 hours, about 4-6 hours, about 6-8, or about 24 hours or longer is provided. In accordance with the present invention, this maybe achieved by inclusion of the anti-connexin polynucleotides (with or without one or more therapeutic agents or agents useful for wound healing) in a formulation together with a pharmaceutically acceptable carrier or vehicle, particularly in the form of a formulation for continuous or slow-release administration.

The routes of administration and dosages described herein are intended only as a guide since a skilled physician will determine the optimum route of administration and dosage for any particular patient and wound.

Any of the methods of treating a subject having or suspected of having or a disease, disorder, and/or wound, referenced or described herein may utilize the administration of any of the doses, dosage forms, formulations, and/or compositions herein described.

### Dressings and Matrices

In one aspect, the one or more anti-connexin polynucleotides alone or in combination with one or more therapeutic polynucleotides and/or polynucleotides useful in wound healing are provided in the form of a dressing or matrix. In certain embodiments, the one or more polynucleotides of the invention are provided in the form of a liquid, semi solid or solid composition for application directly, or the composition is applied to the surface of, or incorporated into, a solid contacting layer such as a dressing gauze or matrix. The dressing composition may be provided for example, in the form of a fluid or a gel. The one or more anti-connexin polynucleotides, alone or in combination with one or more therapeutic polynucleotides, agents useful in wound healing, and/or anti-microtubule agents, may be provided in combination with conventional pharmaceutical excipients for topical application. Suitable carriers include: Pluronic gels, Polaxamer gels, Hydrogels containing cellulose derivatives, including hydroxyethyl cellulose, hydroxymethyl cellulose, carboxymethyl cellulose, hydroxypropylmethyl cellulose and mixtures thereof; and hydrogels containing polyacrylic acid (Carbopols). Suitable carriers also include creams/ointments used for topical pharmaceutical preparations, e.g., creams based on cetomacrogol emulsifying ointment. The above carriers may include alginate (as a thickener or stimulant), preservatives such as benzyl alcohol, buffers to control pH such as disodium hydrogen phosphate/sodium dihydrogen phosphate, agents to adjust osmolarity such as sodium chloride, and stabilizers such as EDTA.

In one embodiment one or more anti-connexin polynucleotides, for example a connexin 43 antisense polynucleotide, preferably a connexin 43 antisense oligodeoxynucleotide, is administered, alone or in combination with one or more therapeutic agents, agents useful in wound healing and/or anti-microtubule agents, on a natural or synthetic matrix.

Suitable dressings or matrices may include, for example, the following with one or more anti-connexin polynucleotides either alone or in combination with one or more therapeutic agents, agents useful in wound healing and/or anti-microtubule agents. An anti-connexin 43 oligonucleotide is preferred, for example an anti-connexin 43 antisense oligonucleotide:

1) Absorptives: suitable absorptives may include, for example, absorptive dressings, which can provide, for example, a semi-adherent quality or a non-adherent layer, combined with highly absorptive layers of fibers, such as for example, cellulose, cotton or rayon. Alternatively, absorptives may be used as a primary or secondary dressing.

2) Alginates: suitable alginates include, for example, dressings that are non-woven, non-adhesive pads and ribbons composed of natural polysaccharide fibers or xerogel derived from seaweed. Suitable alginates dressings may, for example, form a moist gel through a process of ion exchange upon contact with exudate. In certain embodiments, alginate dressings are designed to be soft and conformable, easy to pack, tuck or apply over irregular-shaped areas. In certain embodiments, alginate dressings may be used with a second dressing.

3) Antimicrobial Dressings: suitable antimicrobial dressings may include, for example, dressings that can facilitate delivery of bioactive agents, such as, for example, silver and polyhexamethylene biguanide (PHMB), to maintain efficacy against infection, where this is needed or desirable. In certain embodiments, suitable antimicrobial dressings may be available as for example, as sponges, impregnated woven gauzes, film dressings, absorptive products, island dressings, nylon fabric, non-adherent barriers, or a combination of materials.

4) Biological & Biosynthetics: suitable biological dressings or biosynthetic dressings may include, for example, gels, solutions or semi-permeable sheets derived from a natural source, e.g., pigs or cows. In certain embodiments, a gel or solution is applied to the treatment site and covered with a dressing for barrier protection. In another embodiment, a biological-based (e.g., pig intestinal mucosa or bladder tissue) or biosynthetic-based sheet is placed in situ which may act as membrane, remaining in place after a single application, or the may be biological dressings or biosynthetic dressings may be prepared in advance to include one or more, preferably two, anti-connexin agents.

5) Collagens: suitable collagen dressings may include, for example, gels, pads, particles, pastes, powders, sheets or solutions derived from for example, bovine, porcine or avian sources or other natural sources or donors. In certain embodiments, the collagen dressing may interact with treatment site exudate to form a gel. In certain embodiments, collagen dressing may be used in combination with a secondary dressing.

6) Composites: suitable composite dressings may include, for example, dressings that combine physically distinct components into a single product to provide multiple functions, such as, for example, a bacterial barrier, absorption and adhesion. In certain embodiment, the composite dressings are comprised of, for example, multiple layers and incorporate a semi- or non-adherent pad. In certain embodiment, the composite may also include for example, an adhesive border of non-woven fabric tape or transparent film. In certain other embodiment, the composite dressing may function as for example, either a primary or a secondary dressing and in yet another embodiment, the dressing may be used in combination with topical pharmaceutical composition.

7) Contact Layers: suitable contact layer dressings may include, for example, thin, non-adherent sheets placed on an area to protect tissue from for example, direct contact with other agents or dressings applied to the treatment site. In certain embodiments, contact layers may be deployed to conform to the shape of the area of the treatment site and are porous to allow exudate to pass through for absorption by an overlying, secondary dressing. In yet another embodiment, the contact layer dressing may be used in combination with topical pharmaceutical composition.

8) Elastic Bandages: suitable elastic bandages may include, for example, dressings that stretch and conform to the body contours. In certain embodiment, the fabric composition may include for example, cotton, polyester, rayon or nylon. In certain other embodiments, the elastic bandage may for example, provide absorption as a second layer or dressing, to hold a cover in place, to apply pressure or to cushion a treatment site.

9) Foams: suitable foam dressings may include, for example, sheets and other shapes of foamed polymer solutions (including polyurethane) with small, open cells capable of holding fluids. Exemplary foams may be for example, impregnated or layered in combination with other materials. In certain embodiment, the absorption capability may be adjusted based on the thickness and composition of the foam. In certain other embodiments, the area in contact with the treatment site may be non-adhesive for easy removal. In yet another embodiment, the foam may be used in combination with an adhesive border and/or a transparent film coating that can serve as an anti-infective barrier.

10) Gauzes & Non-Woven dressings: suitable gauze dressings and woven dressings may include, for example, dry woven or non-woven sponges and wraps with varying degrees of absorbency. Exemplary fabric composition may include, for example, cotton, polyester or rayon. In certain embodiment, gauzes and non-woven dressing may be available sterile or non-sterile in bulk and with or without an adhesive border. Exemplary gauze dressings and woven dressings may be used for cleansing, packing and covering a variety of treatment sites.

11) Hydrocolloids: suitable hydrocolloid dressings may include, for example, wafers, powders or pastes composed of gelatin, pectin or carboxymethylcellulose. In certain embodiment, wafers are self-adhering and available with or without an adhesive border and in a wide variety of shapes and sizes. Exemplary hydrocolloids are useful on areas that require contouring. In certain embodiments, powders and pastes hydrocolloids may use used in combination with a secondary dressing.

12) Hydrogels (Amorphous): suitable amorphous hydrogel dressings may include, for example, formulations of water, polymers and other ingredients with no shape, designed to donate moisture and to maintain a moist healing environments and or to rehydrate the treatment site. In certain embodiment, hydrogels may be used in combination with a secondary dressing cover.

13) Hydrogels: Impregnated Dressings: suitable impregnated hydrogel dressings may include, for example, gauzes and non-woven sponges, ropes and strips saturated with an amorphous hydrogel. Amorphous hydrogels may include for example, formulations of water, polymers and other ingredients with no shape, designed to donate moisture to a dry treatment site and to maintain a moist healing environment.

14) Hydrogel Sheets: suitable hydrogel sheets may include for example, three-dimensional networks of cross-linked hydrophilic polymers that are insoluble in water and interact with aqueous solutions by swelling. Exemplary hydrogels are highly conformable and permeable and can absorb varying amounts of drainage, depending on their composition. In certain embodiment, the hydrogel is non-adhesive against the treatment site or treated for easy removal.

15) Impregnated Dressings: suitable impregnated dressings may include, for example, gauzes and non-woven sponges, ropes and strips saturated with a solution, an emulsion, oil, gel or some other pharmaceutically active compound or carrier agent, including for example, saline, oil, zinc salts, petrolatum, xeroform and scarlet red as well as the compounds described herein.

16) Silicone Gel Sheets: suitable silicone gel sheet dressings may include, for example, soft covers composed of cross-linked polymers reinforced with or bonded to mesh or fabric.

17) Solutions: suitable liquid dressings may include, for example, mixtures of multiprotein material and other elements found in the extracellular matrix. In certain embodiment, exemplary solutions may be applied to the treatment site after debridement and cleansing and then covered with an absorbent dressing or a nonadherent pad.

18) Transparent Films: suitable transparent film dressings may include polymer membranes of varying thickness coated on one side with an adhesive. In certain embodiments, transparent films are impermeable to liquid, water and bacteria but permeable to moisture vapor and atmospheric gases. In certain embodiments, the transparency allows visualization of the treatment site.

19) Fillers: suitable filler dressings may include, for example, beads, creams, foams, gels, ointments, pads, pastes, pillows, powders, strands or other formulations. In certain embodiment, fillers are non-adherent and may include a time-released antimicrobial. Exemplary fillers may be useful to maintain a moist environment, manage exudate, and for treatment of for example, partial- and full-thickness wounds, infected wounds, draining wounds and deep wounds that require packing.

Any of the methods of treating a subject having or suspected of having or predisposed to, or at risk for, a disease, disorder, and/or condition, referenced or described herein may utilize the administration of any of the doses, dosage forms, formulations, and/or compositions herein described.

### Treatment

Thus, in accordance with the invention, there are provided formulations by which cell-cell communication can be regulated or downregulated in a transient and site-specific manner, as well as regulation of communication via connexins to the extracellular environment. The formulations therefore have application in methods of therapy and in other treatments.

In instances of tissue damage that may produce adhesions, the formulations of the invention will be effective in both preventing adhesions or decreasing severity and promoting the minimization of adhesions where needed. The formulations therefore will have benefit in the prevention and/or treatment of adhesions, whether the result of external trauma, surgical intervention or disease state, for example.

Utilizing the polynucleotides, compositions and methods provided herein a wide variety of surgical adhesions and complications of surgery can be treated or prevented. Adhesion formation complicates a variety of surgical procedures. As described above, surgical adhesions complicate virtually any open or endoscopic surgical procedure in the abdominal or pelvic cavity.

Thus invention relates to a method of preventing or decreasing post-surgical adhesions in a subject which comprises administering an effective amount of an anti-connexin polynucleotide to the patient at a site of surgery. In one embodiment the anti-connexin polynucleotide is administered at and/or about the site of surgical incision. In one embodiment the anti-connexin polynucleotide is administered during and/or after surgery. In one embodiment the anti-connexin polynucleotide is effective, in whole or in part, to (1) downregulate expression of a connexin protein (2) inhibit intercellular communication by decreasing gap junction formation, (3) prevent or reduce surgical adhesions at a site of the surgery or surgical repair.

It also relates to a method of preventing or decreasing formation of secondary surgical adhesion, comprising administration of an effective amount of an anti-connexin polynucleotide to subject a following a procedure to repair an adhesion. In one embodiment the procedure is a separation or release procedure. In one embodiment the anti-connexin polynucleotide is administered at the site of surgical incision. In one embodiment the anti-connexin polynucleotide is administered during and/or after surgery. In one embodiment the anti-connexin polynucleotide is effective, in whole or in part, to (1) downregulate expression of a connexin protein (2) inhibit intercellular communication by decreasing gap junction formation, (3) prevent or reduce secondary surgical adhesions at a site of the surgery or surgical repair.

In certain embodiments, the anti-connexin polynucleotide is administered to epithelial, connective, muscle, and nerve tissue or other tissue exposed or wounded during surgery or as a result of trauma. In one embodiment, the anti-connexin polynucleotide is administered topically. In other embodiments, the anti-connexin polynucleotide is implanted or instilled or injected.

Thus invention relates to a method of preventing or decreasing injury- or trauma-related adhesions in a subject which comprises administering an effective amount of an anti-connexin polynucleotide to the patient at a site of trauma or injury.

The anti-connexin polynucleotide can be administered in any manner which achieves a desired result. Preferred methods include peritubular administration (either direct application at the time of surgery or with endoscopic, ultrasound, CT, MRI, or fluoroscopic guidance); “coating” the surgical implant; and placement of a drug-eluting polymeric implant at the surgical site. In a preferred embodiment, 0.5% to 20% anti-connexin polynucleotide by weight is loaded into a polymeric carrier (as described in the following examples) and applied to the peritubular (mesenteric) surface as a “paste”, “film”, or “wrap” which releases the drug over a period of time such that the incidence of surgical adhesions is reduced. During endoscopic procedures, the anti-connexin polymer preparation may be applied as a “spray”, via delivery ports in the endoscope, to the mesentery of the abdominal and pelvic organs manipulated during the operation. In a particularly preferred embodiment, the peritubular composition is about 0.1% to about 5% anti-connexin polynucleotide by weight. In another preferred embodiment, a polymeric coating containing about 0.1% to about 20% or more or an anti-connexin polynucleotide is applied to the surface of the surgical implant (e.g., breast implant, artificial joint, vascular graft, etc.) to prevent encapsulation/inappropriate scarring in the vicinity of the implant. In yet another preferred embodiment, a polymeric implant containing about 0.01% to about 20% or more of an anti-connexin polynucleotide by weight is applied directly to the surgical site (e.g., directly into the sinus cavity, chest cavity, abdominal cavity, or at the operative site during neurosurgery) such that recurrence of inflammation, adhesion formation, or scarring is reduced. In another embodiment, lavage fluid containing about 1 to about 100 μg/cm² (preferably about 10 to about 50 μg/cm²) of an anti-connexin polynucleotide, would be used at the time of or immediately following surgery and administered during surgery or intraperitoncally, by a physician. In all of the embodiments, other anti-connexin polynucleotides would be administered at equivalent doses adjusted for potency and tolerability of the polynucleotide.

The invention also relates to a method to evaluate the anti-adhesion activity of an anti-connexin polynucleotide, comprising contacting cells at risk of forming an adhesion with an anti-connexin polynucleotide, and determining the anti-adhesion effect of said an anti-connexin polynucleotide. In one embodiment, said method is carried out in vitro. In another embodiment, said method is carried out in vivo.

A number of animal models have been used to evaluate therapeutic potential for treating adhesions. Two model systems that may be employed are the side wall adhesion model and the uterine horn model and are more fully described in the Examples. These models may be used to determine the potential of an anti-connexin polynucleotide (with or without one or more therapeutic agents, agents useful in wound healing and/or anti-microtubule agents) in preventing or decreasing adhesions. A clear correlation between results obtained using both of these models and utility in adhesion prevention has been demonstrated with INTERCEED (TC7), for which clear clinical efficacy has been shown and FDA approval for adhesion prevention in gynecological surgery has been obtained.

In the peritoneal sidewall model, rabbits are pre-anesthetized with 1.2 mg/kg acetylpromazine and anesthetized with a mixture of 55 mg/kg ketamine hydrochloride and 5 mg/kg xylazine intramuscularly. Following preparation for sterile surgery, a midline laparotomy is performed. A 3×5-cm area of peritoneum and transversus abdominis muscle is removed on the right lateral abdominal wall. The cecum is exteriorized, and digital pressure is exerted to create subserosal hemorrhages over all cecal surfaces. The cecum is then returned to its normal anatomic position. The polynucleotide to be tested is placed in an Alzet miniosmotic pump (Alza Corporation, Palo Alto, Calif., USA) to allow continuous release of the molecule through the postsurgical interval. The Alzet miniosmotic pump is placed in the subcutaneous space and a delivery tube connected the pump with the site of injury at sidewall. Vehicle is placed in the pump of control rabbits. The abdominal wall and skin are closed in a standardized manner. After 7 days, the rabbits are sacrificed and the percentage of the area of the sidewall injury that is involved in adhesions is determined. In addition, the tenacity of the adhesion formed is scored using a system as follows:

0=No adhesions

1=mild, easily dissectable adhesions

2=moderate adhesions; non-dissectable, does not tear organ

3=dense adhesions; non-dissectable, tears when removed

A reduction in the area or the tenacity of the adhesions would be considered beneficial.

In additional experiments, a rabbit uterine horn model may be employed. This model has been previously shown to cause severe adhesions in rabbits after surgery [Nishimura, K. et al., “The Use of Ibuprofen for the Prevention of Postoperative Adhesions in Rabbits,” Am. J. Med., Vol. 77, pp. 102-106 (1984)]. The rabbits are anesthetized (130 mg/kg ketamine and 20 mg/kg acetylpromazine im) and prepared for sterile surgery. A midline laparotomy is performed, and surgical trauma is performed on both uterine horns by abrading the serosal surface with gauze until punctuate bleeding developed. Ischemia of both uterine horns is induced by removal of the collateral blood supply. After traumatization, the abdominal wall is closed in two layers. The polynucleotide to be tested is delivered as described for the peritoneal sidewall model, but the tubing is placed over the injured uterine horns. With the uterine horn model, an initial score to represent the overall extent of adhesions is given (0 to 4+). The percentage of a surface of the horn involved in adhesions to various organs are given in the tables below the overall adhesion score.

### Compositions

The present invention is directed to pharmaceutical compositions and formulations useful in treating or preventing adhesions (e.g. surgical and secondary surgical adhesions), wherein the composition or formulation comprises therapeutically effective amounts of an anti-connexin polynucleotide, such as a connexin antisense polynucleotide.

Equally, in instances of other tissue damage the methods, compositions and formulations of the invention are effective in treating or preventing adhesions. The compositions and formulations, therefore, have clear benefit in the treatment of adhesions.

In one preferred form, the composition contains one or more anti-connexin polynucleotides, for example a connexin antisense polynucleotide, to the mRNA of one connexin protein only. Most preferably, this connexin protein is connexin 43.

Alternatively, the compositions may comprise polynucleotides to more than one connexin protein. Preferably, one of the connexin proteins to which polynucleotides are directed is connexin 43. Other connexin proteins to which oligodeoxynucleotides are directed may include, for example, connexins 26, 30, 31.1, 32, and 37. Suitable exemplary polynucleotides (and ODNs) directed to various connexins are set forth in Table 1.

Many aspects of the invention are described with reference to oligodeoxynucleotides. However it is understood that other suitable polynucleotides (such as RNA polynucleotides) may be used in these aspects. Other anti-connexin oligonucleotides are RNAi and siRNA oligonucleotides.

Accordingly, in one aspect, the invention provides compositions for use in therapeutic treatment for preventing or decreasing occurrence of adhesions, which comprises at least one anti-connexin polynucleotide, preferably an anti-connexin 43 polynucleotide. In a preferred embodiment, the composition further comprises a pharmaceutically acceptable carrier or vehicle.

### Kits, Medicaments and Articles of Manufacturer

In one aspect, the invention provides a kit for preventing or treating adhesions (e.g. surgical and secondary surgical adhesions).

The kit may include one or more compositions described herein. For example, the kit may include a composition comprising an effective amount of one or more anti-connexin polynucleotides, e.g., an anti-connexin 43 polynucleotides, effective for the treatment of a subject having, at risk for, or predisposition to a fibrotic disease, disorder or condition. In one embodiment, the kit comprises a composition that comprises an effective amount of one or more polynucleotide homologues effective for the treatment of a subject having, at risk for, or predisposition to forming adhesions.

Optionally, one or more anti-connexin polynucleotides may also be used in the manufacture of the medicament useful for the treatment of a subject having, at risk for, or predisposition to forming adhesions. In one embodiment, the medicament comprises a therapeutically effective amount of an anti-connexin polynucleotide, preferably an anti-connexin 43 polynucleotide, and a pharmaceutically acceptable carrier.

In another aspect, the invention includes an article of manufacture comprising a vessel containing an effective amount of one or more anti-connexin polynucleotides, e.g., an anti-connexin 43 polynucleotide, and instructions for use, including use for the treatment of a subject having, at risk for, or predisposition to forming adhesions.

A better understanding of the invention will be gained by reference to the following experimental section. The following experiments are illustrative and are not intended to limit the invention or the claims in any way.

### EXAMPLES

### Example 1

An aqueous solution is made of a polyethylenepolyoxypropylene block copolymer having a polyoxypropylene hydrophobe base average molecular weight of about 4000, a total average molecular weight of about 11,500 and containing oxyethylene groups in the amount of about 70% by weight of the total weight of copolymer. This copolymer is sold under the trademark PLUFONIC® F-127 by the BASF Corporation, Parsippany, N.J.

A solution is made by dissolving the polymer in cold (4° C.) distilled water to give a concentration of about 10% to about 30% by weight. More specific solution procedures are described in “Artificial Skin I Preparation and Properties of Pluronic F-127 Gels for Treatment of Burns”, J. Biomed. Mater. Res. 6, 527, 1972. Such solutions are described in U.S. Pat. No. 5,366,735, the disclosure of which is incorporated herein by reference.

### Example 2

The following test procedure is utilized in order to determine the effect of a solution of Example 1 above or the solution of Example 1 including anti-connexin polynucleotide on surgically injured rats, or another formulation. Twenty-two female Sprague-Dawley rats having a 300-400 gram body weight are anesthetized with pentobarbital sodium (30 milligrams per kilogram of body weight) by application intraperitoneally through the left lumbar region of the ventral abdominal wall. The abdomen is thereafter opened by a 5 centimeter midline vertical incision subsequent to cleansing of the abdominal surface with povidone-iodine solution and removing hair by shaving. A one centimeter segment of each uterine horn is stripped of serosa and an opposing one square centimeter of parietal peritoneum is excised, including the underlying muscle layer. Hemostasis may not be attained.

Subsequently, a formulation according to Example 1 is applied at a temperature of 4° C. to both the surgically injured area of the uterine horn and the parietal peritoneum surgical injury but only on one side of the abdomen. After the first application of formulation has formed a gel, a second layer of formulation is applied. Approximately 0.5 to 1.5 cubic centimeters of the formulation is applied depending upon the amount necessary to adequately cover (on one side of the abdomen) both the surgically injured one centimeter sediment of the uterine horn and the surgically injured one square centimeter area of parietal peritoneal tissue.

The remaining side of the abdomen which is surgically injured in the same manner was left untreated. The portion of the uterine horn which is stripped of serosa is then attached within 0.5 centimeter of the surgical injury to the peritoneal parietal area by a single 3-0 VICRYL ligature suture. This is done to insure that the injured surface of the uterine horn remained in close proximity to the surgical injury of the parietal area of the peritoneum until re-peritonealization had occurred. The abdominal wall is closed with a single layer of interrupted 0-0 VICRYL suture and 21 days later each animal is sacrificed and the abdomen was examined for the presence of adhesions.

The following grading system is used to evaluate the results obtained:

0=no adhesions observed.
1=adhesions on 25% of the surgically injured area.
2=adhesions on 50% of the surgically injured area.
3=adhesions on 100% of the surgically injured area.

The tenacity of the adhesion which formed is evaluated according to the following grading system:

0.0=no resistance to separation.
0.5=moderate force of separation required to rupture the adhesion.
1.0=strong force or cutting necessary for separation.

A rating for the results obtained is obtained by adding the results in each of the grading systems. Results therefore would range from 0.0 to 4.0 for each surgically injured area evaluated. The data are analyzed by a rank sum test and also by analysis of variance.

Since the bilaterally surgically injured areas of each rat are treated with block copolymer solution or block copolymer solution with anti-connexin polynucleotide only unilaterally, each rat served as its own control.

The surviving animals are evaluated to determine those that developed adhesions on the untreated control side of the abdomen. Of the 20 surviving rats, the degree of adhesion is noted. The combined score, for the block copolymer treated areas including area and tenacity of adhesions is evaluated.

### Example 3

### A. Preparation of Chitosan Film

5 g hydrochloride salt of Chitosan (20% degree of acetylation, Pronova) are dissolved in a 2% acetic acid solution (0.5 L., 1% v/w). The solution is autoclaved for 1 h at 125° C. for sterilization purposes. After cooling a film is made in a Petri dish, in this case with the use of 20 mL of the solution. The film is then allowed to dry at room temperature and neutralized by the addition of a sodium phosphate buffer, 0.2 M, pH 9.0, added to the dish. The film is allowed to stay in this buffer for 2-4 h at room temperature, is then washed with distilled water 3-4 times and again allowed to dry.

### B. Alternate Preparation of Chitosan Film

5 g hydrochloride salt of chitosan (45% degree of acetylation, Pronova) are dissolved in water (0.5 L, 1% v/w). The solution is autoclaved for 1 h at 125° C. for sterilization purposes. After cooling a film is made in a Petri dish, in this case with the use of 20 mL of the solution. The film is then allowed to dry at room temperature and neutralized by the addition of a sodium phosphate buffer, 0.2 M, pH 9.0, added to the dish. The film is allowed to stay in this buffer for 2-4 h at room temperature, is then washed with distilled water 3-4 times and again allowed to dry.

### C. Preparation of Chitosan Film with Ionically Bonded Test Polynucleotide

5 g hydrochloride salt of chitosan (45% degree of acetylation, Pronova) are dissolved in water (0.5 L, 1% v/w). The solution is autoclaved for 1 h at 125° C. for sterilization purposes. After cooling a film is made in a Petri dish, in this case with the use of 20 mL of the solution. The film is then allowed to dry at room temperature and a solution of anti-connexin test polynucleotide (125 g in 0.5 L water, for example) is added. After 3 hours at room temperature the film is rinsed with 2×0.5 L water and dried.

### Example 4

A film prepared in accordance with Example 3 is used as an anti-adherence membrane in the following animal model. The abdominal wall of a rat is opened and on each side of the sagittal line there is produced in a surgical manner a wound about 12×10 mm. One defect is covered with a film from Example 3, a piece of about 18×15 mm, whereas as the other defect is left open. The membrane is sutured using Dexon® 7-0 in such a manner that no suture is exposed in the abdominal cavity.

The result is evaluated after 2 and 4 weeks. The abdominal defect beneath the film heals essentially with scar tissue formation, and there are signs of inflammatory reaction and capsule formation around the film.

### Example 5

The film made in accordance with Example 3C is used as an anti-adherence membrane in the following animal model.

The abdominal wall of a rat is opened and on each side of the sagittal line there is created in a surgical manner a wound of about 12×10 mm. One defect is covered with film, about 18×15 mm, whereas the other defect is left open. The membrane is sutured in the same manner as in Example 4.

The wound area left open displayed several adherences in contrast to the wound covered by the film, which had very few if any adherences.

### Example 6

Films prepared from chitosan anti-connexin polynucleotide as described above in Example 3C are positioned to cover wounds (10×12 mm, depth 1 mm) prepared on the parietal abdominal wall as described above. An identical wound is prepared on the contralateral side of the abdominal wall, and covered by a Chitosan film as described in Example 3A or B. The occurrence of adherence formation is evaluated after 2 weeks. Light microscopic examination of the film is used to evaluate healing of the wound, including the extent of covering by mesothelial-like cells, and infiltration of inflammatory cells at the interface between the film and the wounded abdominal wall tissue.

### Example 7

Female Sprague Dawley rats, weighing between 175 and 225 grams each, are used in this study. The rats are quarantined at least two days prior to surgery. The rats are housed in a vivarium on a 12:12 hour light/dark cycle. Food and water are available ad libitum except in the immediate postoperative period.

The rats undergo a standardized procedure for laparotomy (intramuscular anesthesia with ketamine/rompum, shaving with animal clippers, betadine scrub, alcohol scrub). A 2 cm incision was then made on the midline. A double-walled gelatin capsule is placed on the right side of the abdomen through the incision. The anti-connexin polynucleotide is administered (e.g., 1-10 and up to about 100 μg/kg/day) for 1-3 days, or 1-3 hours, prior to surgery, and then at various times as desired for 11 days until necropsy. The abdominal wall and skin is then sutured closed using two layers of 4-0 Ethilon suture. Following surgery, the rats receive analgesic for three days and are observed twice daily for signs of morbidity and mortality.

Upon gross observation following an 11 day post-operative observation period, wound closure is evaluated, and the animals evaluated for scarring.

### Example 8

Multiple studies are performed to evaluate or quantitate the efficacy of the active polynucleotides alone or in combination with an anti-adhesion polynucleotide in the redaction of adhesion formation following peritoneal surgery. Two model systems are employed: the sidewall adhesion model and the uterine horn model. A clear correlation between results obtained using both of these models and utility in adhesion prevention has been demonstrated with INTERCEED (TC7), for which clear clinical efficacy has been shown and FDA approval for adhesion prevention in gynecological surgery has been obtained.

### A. Rabbit Sidewall Model

In the peritoneal sidewall model, rabbits are pre-anesthetized with 1.2 mg/kg acetylpromazine and anesthetized with a mixture of 55 mg/kg ketamine hydrochloride and 5 mg/kg xylazine intramuscularly. Following preparation for sterile surgery, a midline laparotomy is performed. A 3×5-cm area of peritoneum and transversus abdominis muscle is removed on the right lateral abdominal wall. The cecum is exteriorized, and digital pressure is exerted to create subserosal hemorrhages over all cecal surfaces. The cecum is then returned to its not anatomic position. The anti-connexin polynucleotide or composition thereof to be tested is placed in an Alzet miniosmotic pump (Alza Corporation, Palo Alto, Calif., USA) to allow continuous release of the molecule through the postsurgical interval. The Alzet miniosmotic pump is placed in the subcutaneous space and a delivery tube connected the pump with the site of injury at sidewall. Vehicle is placed in the pump of control rabbits. The abdominal wall and skin are closed in a standardized manner.

After 7 days, the rabbits are sacrificed and the percentage of the area of the sidewall injury that is involved in adhesions is determined. In addition, the tenacity of the adhesion formed is scored using a system as follows:

0=No adhesions

1=mild, easily dissectable adhesions

2=moderate adhesions; non-dissectable, does not tear organ

3=dense adhesions; non-dissectable, tears when removed

A reduction in the area or the tenacity of the adhesions is considered beneficial.

### B. Rabbit Uterine Horn Model

In additional experiments, a rabbit uterine horn model is employed. This model has been previously shown to cause severe adhesions in rabbits after surgery [Nishimura, K. et al., “The Use of Ibuprofen for the Prevention of Postoperative Adhesions in Rabbits,” Am. J. Med., Vol. 77, pp. 102-106 (1984). The rabbits are anesthetized (130 mg/kg ketamine and 20 mg/kg acetylpromazine im) and prepared for sterile surgery. A midline laparotomy is performed and both uterine horns are surgically traumatized by abrading the serosal surface with gauze until punctuate bleeding develops. Ischemia of both uterine horns is induced by removal of the collateral blood supply. In some studies, the materials re delivered to the site of injury via Alzet miniosmotic pumps and tubes as, described above. In other studies, a portion of the test compositions are applied at the site of injury at the end of surgery and any remaining material is applied through the incision site prior to closing. Controls include surgical and vehicle controls. The abdominal wall and skin are closed in a standardized manner.

After 7 days, the rabbits are sacrificed and the percentage of the area of the uterine born injury that is involved in adhesions is determined. An initial score to represent the overall extent of adhesions is given (0 to 4+). The percentage of a surface of the horn involved in adhesions to various organs is then determined.

### Example 9

The use of anti-connexin polynucleotide loaded PCL film to reduce adhesion is examined in the rabbit uterine horn model.

### A. Methods

The rabbit uterine horn model is conducted essentially as described by Wiseman et al., 1992 (Journal of Reproductive Medicine, 37:766-770), with hemostasis. New Zealand female white rabbits are anesthetized and a mid-line incision made through the skin and the abdominal wall. Both uterine horns are located and exteriorized. Using a French Catheter Scale, the diameter of each uterine horn is measured and recorded. Only those rabbits with uterine horns measuring size 8 to 16, inclusive, on the French scale are used. Using a number 10 scalpel blade, 5 cm lengths of each uterine horn, approximately 1 cm from the uterine bifurcation, are scraped, 40 times per side, until punctuate bleeding. Hemostasis is achieved by tamponade.

Animals are randomized to receive: no treatment (Surgical Control); polymer Vehicle Control; anti-connexin polynucleotide (0.1% in vehicle); and anti-connexin polynucleotide (0.001-1% in vehicle). Test polynucleotide (0.4 to 2.5 ml) is applied over the horns via an 18 gauge needle. Uterine horns are replaced into the pelvis and the abdominal incision closed.

At 18, 31, 32, 33 and 60 days after surgery, animals are euthanized by intravenous injection of sodium pentobarbital (120 mg/ml; 1 ml/kg). Body weights of the animals are recorded. The abdomen is opened and the surgical site inspected. Adhesions are graded by a blinded observer as follows:

### Extent of Adhesions

The total length (cm) of each uterine horn involved with adhesions is estimated and recorded.

### Tenacity of Adhesions

Adhesions are graded as 0 (absent), 1.0 (filmy adhesions) and 2.0 (tenacious, requiring sharp dissection).

### Degree of Uterine Convolution

The degree of uterine convolution is recorded according to the following scale:

No convolution: Straight lengths of adherent or non-adherent horns which are clearly discerned.

Party convoluted: Horns have adhesions and 50%-75% of the horn length is entangled preventing discernment of straight portions.

Completely convoluted: It is impossible to discern uterine anatomy because the horn is completely entangled.

### Example 10

Five-week-old female hamsters (10 hamsters per each group) are anesthetized by administering intraperitoneally pentobarbital sodium (50 mg/kg) and, after midline incision at abdominal region, the uterus is rubbed with a cotton swab. Thereafter, 1 mL of saline solution of a test polynucleotide (e.g., 1−50×10⁻⁴ to 10⁻⁶ mol/L) is added dropwise intraperitoneally, and then the incised part was sutured. On the other hand, as a control, saline alone is added dropwise, followed by a similar treatment.

After 4 weeks from the operation, the animals are euthanized, the abdominal part is exposed and adhesion was investigated. The adhesion is judged using the following 5-grade scoring system and the data are analyzed according to Mann-Whitney U test.

Adhesion Score

0: No adhesions

1: Very weak adhesion (film-like adhesion easily releasable)

2: Limited adhesion (strong adhesion difficult to release at only one point)

3: Wide-range adhesion (strong adhesion difficult to release at several points)

4: Very strong adhesion (very strong adhesion impossible to release)

### Example 11

Six-week-old SD rats are subjected to midline incision at lower abdominal region under pentobarbital anesthetization (70 mg/kg, intramuscular injection), and the cecum is taken out of the incised part. Two parts of serous membrane of the cecum (about 2 cm² each) are rubbed with a cotton swab a hundred times until petechial hemorrhage occurs, followed by dropwise addition of 100 μL of ethanol. The cecum is again set in abdominal cavity, and then, 2 mL of a phosphate buffered saline (hereinafter, abbreviated as PBS, pH 7.4) solution of a test anti-connexin polynucleotide is added dropwise intraperitoneally, and then the incised part is sutured. The concentration of each test polynucleotide solution is as desired. In a control group, PBS alone is added dropwise, followed by a similar treatment. Each group has 11 or 12 rats. After 1 week from the operation, the animals are euthanized, the abdominal part is re-incised and an adhesion state of the cecum is evaluated according to adhesion scores using the adhesion intensity and adhesion area as indexes. The score values are determined according to the following 5-grade scores. In this connection, adhered region (%) is determined as percentage of total area of the adhered parts relative to the area of the rubbed regions.

### Adhesion Score

0: No adhesions
1: Easily releasable adhesion limited to only a part (less than 25% of adhered region)
2: Easily releasable adhesion over a wide range (25% or more of adhered region) or limited adhesion to only a part (less than 25% of adhered region) difficult to release
3: Wide-range adhesion (25% or more of adhered region) difficult to release
4: Adhesion impossible to release or adhesion accompanied by serous membrane injury at release

### Example 12

A beagle dog is anesthetized and each conjunctiva of both eyes thereof is peeled in a size of 10 mm×5 mm under a stereomicroscope. At that time, the tendon is left intact at conjunctival side and but at scleral side. After a sponge immersed in a saline solution of anti-connexin test polynucleotide is placed at the incised part for 3 minutes, the incised part is put in one stitch with 10-0 nylon thread. The concentration of the test polynucleotide solution is as desired and vehicle or saline is used in a control group (6 dogs per each group).

After 7 days from the operation, the animals are euthanized, the eyeballs are taken out and adhesion is investigated. After the thread used at the stitching in the model preparation is cut, evaluation was carried out by pulling the conjunctiva part with tweezers and scoring the adhesion state. The score values are determined according to the following 5-grade scores, and Mann-Whitney U test was used for analyzing the data.

### Adhesion Score

0: No adhesions

1: Very weak adhesion (film-like adhesion easily releasable)

2: Limited adhesion (strong adhesion difficult to release at only one point)

3: Wide-range adhesion (strong adhesion difficult to release at several points)

4: Very strong adhesion (very strong adhesion impossible to release)

The present invention is not limited by the aforementioned particular preferred embodiments. It will occur to those ordinarily skilled in the art that various modifications may be made to the disclosed preferred embodiments with-out diverting from the concept of the invention. All such modifications are intended to be within the scope of the present invention.

### Example 13

Anti-connexin agent is conveniently formulated in a form suitable for administration according to the methods of the present invention.

Suitable formulations include a mixture of the following formulating agents. The amount of the individual anti-connexin agent or agents and formulating agents will depend on the particular use intended.

ASO in PBS
Polyquarternium 10
HEC/HPMC/CMC
Na Hyaluronate
Tween 20
Poloxamer 188
Pluronic 87 NF
SLES
Poly L-lysine/Polyethylene Imine
Banzalkonium chloride
Methylparaben
Proplparaben
Propylene Glycol
10 mM Phosphate Buffer

### Example 14

Formulations for use according to methods of the present invention are prepared by mixing the compounds in the proportions noted below. In one preferred embodiment, the anti-connexin agent is an anti-connexin polynucleotide. In other embodiments, the anti-connexin polynucleotide is an anti-sense oligonucleotide, for example, an anti-sense oligonucleotide of SEQ. ID. NO. 1

### Formulation A

Made up of the following materials (% w/w)—Anti-connexin agent in phosphate-buffered saline (0.47%); Methylparaben (0.17%); Propylparaben (0.03%); Propylene Glycol (1.5%); HPMC (1.5%); and 10 mM Phosphate Buffer (96.33%). Formulation is a clear gel with pH ˜6.74 and osmolality of 244.

### Formulation B

Made up of the following materials (% w/w)—Anti-connexin agent in phosphate-buffered saline (0.47%); Methylparaben (0.17%); Propylparaben (0.03%); Propylene Glycol (1.5%); HPMC (1.5%); 0.5% BAC (0.1%); and 10 mM Phosphate Buffer (96.23%). Formulation is a clear gel with pH ˜6.65 and osmolality of 230.

### Formulation C

Made up of the following materials (% w/w)—Anti-connexin agent in phosphate-buffered saline (0.47%); Methylparaben (0.17%); Propylparaben (0.03%); Propylene Glycol (1.5%); HPMC (1.5%); Polyquaternium 10 (0.5%); Poloxamer 188 (0.1%); and 10 mM Phosphate Buffer (95.73%). Formulation is a slightly hazy gel with pH ˜6.59 and osmolality of 233.

### Formulation D

Made up of the following materials (% w/w)—Anti-connexin agent in phosphate-buffered saline (0.47%); Methylparaben (0.17%); Propylparaben (0.03%); Propylene Glycol (1.5%); HPMC (1.5%); SLES (0.5%); and 10 mM Phosphate Buffer (95.83%). Formulation is a clear gel with pH ˜6.8 and osmolality of 246.

### Formulation E

Made up of the following materials (% w/w)—Anti-connexin agent in phosphate-buffered saline (0.47%); Methylparaben (0.17%); Propylparaben (0.03%); Propylene Glycol (1.5%); HPMC (1.5%); Poloxamer 188 (0.1%); 25K Polyethylene Imine (0.075%); and 10 mM Phosphate Buffer (96.155%). Formulation is a hazy gel with pH ˜7.8 and osmolality of 249.

### Formulation F

Made up of the following materials (% w/w)—Anti-connexin agent in phosphate-buffered saline (0.47%); Methylparaben (0.17%); Propylparaben (0.03%); Propylene Glycol (1.5%); HPMC (1.5%); Sodium Hyaluronate (0.1%); and 10 mM Phosphate Buffer (96.23%). Formulation is a clear gel with pH ˜6.88 and osmolality of 289.

All patents, publications, scientific articles, web sites, and other documents and materials referenced or mentioned herein are indicative of the levels of skill of those skilled in the art to which the invention pertains, and each such referenced document and material is hereby incorporated by reference to the same extent as if it had been incorporated by reference in its entirety individually or set forth herein in its entirety. Applicants reserve the right to physically incorporate into this specification any and all materials and information from any such patents, publications, scientific articles, web sites, electronically available information, and other referenced materials or documents.

The written description portion of this patent includes all claims. Furthermore, all claims, including all original claims as well as all claims from any and all priority documents, are hereby incorporated by reference in their entirety into the written description portion of the specification, and Applicants reserve the right to physically incorporate into the written description or any other portion of the application, any and all such claims. Thus, for example, under no circumstances may the patent be interpreted as allegedly not providing a written description for a claim on the assertion that the precise wording of the claim is not set forth in haec verba in written description portion of the patent.

The claims will be interpreted according to law. However, and notwithstanding the alleged or perceived ease or difficulty of interpreting any claim or portion thereof, under no circumstances may any adjustment or amendment of a claim or any portion thereof during prosecution of the application or applications leading to this patent be interpreted as having forfeited any right to any and all equivalents thereof that do not form a part of the prior art.

All of the features disclosed in this specification may be combined in any combination. Thus, unless expressly stated otherwise, each feature disclosed is only an example of a generic series of equivalent or similar features.

It is to be understood that while the invention has been described in conjunction with the detailed description thereof, the foregoing description is intended to illustrate and not limit the scope of the invention, which is defined by the scope of the appended claims. Thus, from the foregoing, it will be appreciated that, although specific embodiments of the invention have been described herein for the purpose of illustration, various modifications may be made without deviating from the spirit and scope of the invention. Other aspects, advantages, and modifications are within the scope of the following claims and the present invention is not limited except as by the appended claims.

The specific methods and compositions described herein are representative of preferred embodiments and are exemplary and not intended as limitations on the scope of the invention. Other objects, aspects, and embodiments will occur to those skilled in the art upon consideration of this specification, and are encompassed within the spirit of the invention as defined by the scope of the claims. It will be readily apparent to one skilled in the art that varying substitutions and modifications may be made to the invention disclosed herein without departing from the scope and spirit of the invention. The invention illustratively described herein suitably may be practiced in the absence of any element or elements, or limitation or limitations, which is not specifically disclosed herein as essential. Thus, for example, in each instance herein, in embodiments or examples of the present invention, the terms “comprising”, “including”, “containing”, etc. are to be read expansively and without limitation. The methods and processes illustratively described herein suitably may be practiced in differing orders of steps, and that they are not necessarily restricted to the orders of steps indicated herein or in the claims.

The terms and expressions that have been employed are used as terms of description and not of limitation, and there is no intent in the use of such terms and expressions to exclude any equivalent of the features shown and described or portions thereof, but it is recognized that various modifications are possible within the scope of the invention as claimed. Thus, it will be understood that although the present invention has been specifically disclosed by various embodiments and/or preferred embodiments and optional features, any and all modifications and variations of the concepts herein disclosed that may be resorted to by those skilled in the art are considered to be within the scope of this invention as defined by the appended claims.

The invention has been described broadly and generically herein. Each of the narrower species and subgeneric groupings falling within the generic disclosure also form part of the invention. This includes the generic description of the invention with a proviso or negative limitation removing any subject matter from the genus, regardless of whether or not the excised material is specifically recited herein.

It is also to be understood that as used herein and in the appended claims, the singular forms “a,” “an,” and “the” include plural reference unless the context clearly dictates otherwise, the term “X and/or Y” means “X” or “Y” or both “X” and “Y”, and the letter “s” following a noun designates both the plural and singular forms of that noun. In addition, where features or aspects of the invention are described in terms of Markush groups, it is intended, and those skilled in the art will recognize, that the invention embraces and is also thereby described in terms of any individual member and any subgroup of members of the Markush group, and applicants reserve the right to revise the application or claims to refer specifically to any individual member or any subgroup of members of the Markush group.

Other embodiments are within the following claims. The patent may not be interpreted to be limited to the specific examples or embodiments or methods specifically and/or expressly disclosed herein. Under no circumstances may the patent be interpreted to be limited by any statement made by any Examiner or any other official or employee of the Patent and Trademark Office unless such statement is specifically and without qualification or reservation expressly adopted in a responsive writing by Applicants.

What is claimed is:

1. A method of preventing or decreasing adhesion formation in a subject which comprises administering to a patient in need of thereof a therapeutically effective amount of an anti-connexin polynucleotide.

2. The method of claim 1, wherein the anti-connexin polynucleotide decreases connexin protein expression, wherein said connexin is selected from the group consisting of connexin 26, connexin 30, connexin 30.3, connexin 31.1, connexin 32, connexin 36, connexin 37, connexin 40, connexin 40.1, connexin 43, connexin 45, connexin 46 and connexin 46.6.

3. The method of claim 2 wherein the anti-connexin polynucleotide is an antisense oligonucleotide.

4. A method according to claim 3 where the anti-connexin polynucleotide decreases expression of a connexin selected from connexin 26, connexin 30, connexin 31.1, connexin 32, connexin 36, connexin 37, connexin 40, and connexin 45.

5. A method according to claim 3 wherein the anti-connexin polynucleotide decreases expression of connexin 43.

6. The method of claim 2 wherein the anti-connexin polynucleotide is an siRNA or an RNAi oligonucleotide.

7. The method of claim 1, wherein the tissue is selected from the group consisting of epithelial, connective, muscle, and nerve tissue.

8. The method of claim 1 wherein said subject has undergone or is undergoing a surgical procedure.

9. The method of claim 8 wherein the anti-connexin polynucleotide is administered to prevent or reduce surgical adhesion formation.

10. The method of claim 8 wherein the anti-connexin polynucleotide is administered at the site of a surgical opening.

11. The method of claim 8 wherein the anti-connexin polynucleotide is administered at an internal surgical site.

12. The method of claim 1, wherein the anti-connexin polynucleotide inhibits intercellular communication by decreasing gap junction formation.

13. The method of claim 2, wherein the connexin is a human connexin.

14. A method of preventing or decreasing formation of surgical adhesions in a patient at risk thereof, which comprises administering a therapeutically effective amount of an anti-connexin polynucleotide to said patient.

15. The method of claim 14 wherein said patient has had or is undergoing a surgery.

16. A method according to claim 14, wherein said method comprises administering an amount of an anti-connexin oligonucleotide to said patient that is effective to block or inhibit adhesion formation.

17. A method according to claim 16 wherein the anti-connexin oligonucleotide is an anti-connexin 43 oligonucleotide.

18. A method according to claim 14 wherein said anti-connexin polynucleotide is administered topically.

19. A method according to claim 14 wherein said anti-connexin polynucleotide is implanted or instilled.

20. A method according to claim 1 or 14 wherein the anti-connexin polynucleotide is oligonucleotide is selected from the group consisting of SEQ.ID.NOS: 3 to 12.

21. A method according to claim 1 or 14 wherein the connexin oligonucleotide is selected from SEQ. ID. NOS. 1 and 2.

22. A method of preventing or decreasing formation of secondary surgical adhesion, comprising administration of an effective amount of an anti-connexin polynucleotide to subject a following a procedure to repair an adhesion.

23. A method of claim 22 wherein the procedure is a separation or release procedure.

24. A method of claim 22 wherein the anti-connexin polynucleotide is administered at the site of surgical incision.

25. A method of claim 22 wherein the anti-connexin polynucleotide is administered during and/or after surgery.

26. A method of claim 22 wherein the anti-connexin polynucleotide is effective to downregulate expression of a connexin protein at the site of administration, in whole or in part.

27. A method of claim 26 wherein the anti-connexin polynucleotide decreases expression of connexin 43.

28. The method of claim 22 wherein the anti-connexin polynucleotide is an anti-connexin 43 oligonucleotide.

29. The method of claim 22 wherein the anti-connexin polynucleotide is a connexin 43 antisense oligonucleotide.

30. The method of claim 22 wherein the anti-connexin polynucleotide is an siRNA or an RNAi oligonucleotide.

31. A method of claim 22 wherein the anti-connexin polynucleotide is effective to inhibit intercellular communication by decreasing gap junction formation, in whole or in part.

32. A method of claim 22 wherein the anti-connexin polynucleotide is effective to prevent or reduce secondary surgical adhesions at a site of the surgery or surgical repair, in whole or in part.

33. A lavage solution which comprises an anti-connexin polynucleotide.

34. The lavage solution according to claim 33 wherein said lavage solution is formulated for arthroscopic lavage, for bronchioalveolar lavage, gastric lavage, peritoneal lavage, or ductal lavage.

35. The lavage solution according to claim 33 wherein said anti-connexin polynucleotide is an anti-connexin 43 oligonucleotide.

36. The lavage solution according to claim 33 wherein said anti-connexin polynucleotide is a connexin 43 antisense oligonucleotide.

37. The lavage solution according to any of claims 33-36 wherein the anti-connexin polynucleotide reduces connexin protein expression.

38. An article of manufacture comprising: (a) a pharmaceutical composition having (i) an anti-connexin polynucleotide in an amount effective to prevent adhesions, and (ii) a pharmaceutically acceptable carrier, and (b) instructions for administering the pharmaceutical composition to a patient who has had or is having a surgery.

39. The article of claim 38 wherein the instructions describe administration of the pharmaceutical composition to the patient to reduce or prevent surgical adhesions after a surgical procedure and administering the pharmaceutical composition in a quantity sufficient to prevent or reduce surgical adhesions at a site of the procedure or a resulting wound.

40. A method of making an article of manufacture, which method comprises: combining (a) a container including a pharmaceutical composition comprising (i) an anti-connexin polynucleotide in an amount effective to prevent adhesions, and (ii) a pharmaceutically acceptable carrier, and (b) labeling instructions for treating a patient having or at risk of having a surgical adhesion by administering the pharmaceutical composition to a patient having a surgical procedure.

41. The method of claim 40 wherein the instructions describe administration of the pharmaceutical composition to the patient and administering the pharmaceutical composition in a quantity sufficient to prevent or reduce adhesions at a site of the surgery.

42. A method of making an article of manufacture, which method comprises: combining (a) a container including a lavage solution comprising (i) an anti-connexin polynucleotide in an amount effective to prevent adhesions e, and (ii) a pharmaceutically acceptable lavage solution, and (b) labeling instructions for administering the lavage solution to a patient during a surgical procedure.

43. The method of claim 42, wherein the anti-connexin polynucleotide decreases connexin 43 protein expression.

44. The method of claim 42 wherein the anti-connexin polynucleotide is an antisense oligonucleotide.

45. The method of claim 42 wherein the anti-connexin polynucleotide is a connexin 43 antisense oligonucleotide.

46. The method of claim 42 wherein the anti-connexin polynucleotide inhibits intercellular communication by decreasing gap junction formation.

47. The method of any of claim 1, 14, or 22 wherein the connexin is a human connexin.

48. The article of any of claim 38, 40 or 42 wherein the connexin is a human connexin.

49. The method of any of claim 1, 14 or 22, wherein the patient or subject is a human.

50. The method of any of claim 1, 14 or 22, wherein the patient or subject is a non-human animal.

51. The method of claim 50, wherein the non-human animal is a sports or pet animal.

52. The method of claim 50, wherein the non-human animal is a horse, a dog, or a cat.
